(19) United States
(12) Patent Application Publication
(10) Pub. No.: US 2005/0222136 A1
(43) Pub. Date: Oct. 6, 2005

(54) Active substance combination

(76) Inventors: Helmut Heinrich Buschmann, Esplugues de Llobregat (ES); Bonifacio Gutierrez Silva, Barcelona (ES); Jorg Holenz, Vilanova i la Geltru (ES); Antonio Farre Gomis, Barcelona (ES)

Correspondence Address: PERMAN & GREEN, 425 POST ROAD, FAIRFIELD, CT, 06824, US

(21) Appl. No.: 10/987,803

(22) Filed: Nov. 12, 2004

(30) Foreign Application Priority Data
Apr. 5, 2004 (ES) 2004 00844

(51) Int. Cl. A61K 31/551; A61K 31/4164; A61K 31/416; A61K 31/415

(52) U.S. Cl. 514/221; 514/396; 514/406

(57) ABSTRACT

The present invention relates to an active substance combination including at least one substituted carbinol compound and at least one non-steroidal anti-inflammatory drug (NSAID), a medicament including the active substance combination, a pharmaceutical formulation including the active substance combination and the use of the active substance combination for the manufacture of a medicament.

The present invention relates to an active substance combination comprising at least one substituted carbinol compound and at least one non-steroidal anti-inflammatory drug (NSAID), a medicament comprising said active substance combination, a pharmaceutical formulation comprising said active substance combination and the use of said active substance combination for the manufacture of a medicament.

Non-steroidal anti-inflammatory drugs such as acetylsalicylic acid or diclofenac are regularly used for the treatment of mild to moderate pain and fever. The analgesic action of this class of compounds results from their inhibition of the enzymatic production of prostaglandins.

Cyclooxygenase is the key enzyme in the conversion of the of arachidonic acid derived from lipids of the cell membrane to prostaglandins and other eicosanoids. Cyclooxygenase exists in two different isoforms characterized by different expression patterns. Cyclooxygenase-1 is constitutively expressed in many cells of the body and responsible mainly for the production of eicosanoids serving normal physiological functions.

Cyclooxygenase-2 expression is induced during inflammation and is considered to be responsible for the production of eicosanoids serving normal physiological functions in a healthy organism.

Many non-steroidal anti-inflammatory drugs have been developed, which show inhibition of Cyclooxygenase-1 and/or inhibition of Cyclooxygenase-2. However, the administration of medicaments comprising such compounds to patients is regularly accompanied by undesired side effects.

Typical side effects associated with the administration of compounds showing Cyclooxygenase-1 specifity or balanced Cyclooxygenase-1 and Cyclooxygenase-2 inhibition are gastrointestinal side effects such as damage of the gastric mucosa.

Although to a lesser extent these side effects are also encountered in the therapy with Cyclooxygenase-2 inhibitors of the first generation, i.e. compounds which show a stronger inhibition of Cyclooxygenase-2 compared to Cyclooxygenase-1.

Whereas undesired gastrointestinal side effects are further reduced if inhibitors with even higher selectivity for Cyclooxygenase-2 are used in the therapy such so-called Cyclooxygenase-2 inhibitors of the second or higher generation are accompanied by other undesired side effects, particularly an increased risk of cardiovascular diseases such as edema, hypertonia or tachycardia.

It was therefore an object of the present invention to provide a medicament having similar or even improved pharmacological efficacy, particularly analgesic efficacy, compared to medicaments comprising non-steroidal anti-inflammatory drugs (NSAIDS) known from the prior art. Preferably said medicament should not show the undesired side effects of such medicaments known from the prior art, or at least less frequent and/or to a lesser extent.

It has now surprisingly been found that similar or improved pharmacological efficacy, particularly analgesic efficacy, is achieved, if one or more non-steroidal anti-inflammatory drugs are administered in combination with one or more substituted carbinol compounds of general formula I given below.

Consequently, the dose of the NSAID component to be administered may be reduced and undesired side effects typically associated with the administration of such compounds occur less frequently and/or in less pronounced form.

Thus, in one of its aspects the present invention relates to an active substance combination comprising
(A) at least one substituted carbinol compound of general formula I, 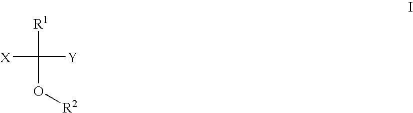
   wherein
R¹ represents a hydrogen atom, a linear or branched alkyl radical, a linear or branched alkenyl radical, an optionally at least mono-substituted cycloaliphatic radical, which may contain at least one nitrogen atom as ring member, or a phenyl radical,
R² represents a hydrogen atom, an optionally at least one nitrogen atom as ring member containing cycloaliphatic radical, which may be at least mono-substituted by a linear or branched alkyl radical and/or which may be bound via a linear or branched alkylene group, a NR³R⁴-moiety, which is bound via a linear or branched alkylene group, or a NR⁵R⁶-moiety, which is bound via a linear or branched alkylene group,
R³ and R⁴, identical or different, represent a linear or branched alkyl radical or an unsubstituted benzyl radical,
R⁵ and R⁶ together with the bridging nitrogen atom represent a saturated, unsubstituted, optionally at least one further heteroatom as ring member containing heterocyclic radical,
X represents an optionally at least mono-substituted phenyl radical or an optionally at least mono-substituted thienyl radical, wherein in each case the substituents are selected from the group consisting of a linear or branched alkyl radical, a linear or branched alkoxy group, a linear or branched alkyl radical, which is at least partially halogenated or a halogen atom,
Y represents a heteroaryl radical, which contains one or more nitrogen atoms as ring members and which is unsubstituted or at least mono-substituted by one or more substitutents independently from one another selected from the group consisting of a halogen atom, a linear or branched alkyl radical, an unsubstituted benzyl radical, a ciano group bound via a linear or branched C₁₋₄-alkylene group, a carboxy group bound via a linear or branched C₁₋₄-alkylene group, a methoxy carbonyl group bound via a linear or branched C₁₋₄-alkylene group, a hydroxy group bound via a linear or branched C₁₋₄-alkylene group, an amino group bound via a linear or branched C₁₋₄-alkylene group, a (C₁₋₄) dialkylamino group bound via a linear or branched C₁₋₄-alkylene group and a cycloaliphatic radical, which contains one or more nitrogen atoms as ring members and which is bound via a linear or branched C₁₋₄-alkylene group, or Y represents an unsubstituted heteroaryl radical, which contains two nitrogen atoms as ring members and which is condensed with (annellated to) a saturated, one methyl-substituted nitrogen atom as ring member containing cycloaliphatic group,
optionally in form of one of its stereoisomers, preferably enantiomers or diastereomers, its racemate or in form of a mixture of at least two of its stereoisomers, preferably enantiomers and/or diastereomers, in any mixing ratio, or a corresponding physiologically acceptable salt thereof, or a corresponding solvate, and
(B) at least one non-steroidal anti-inflammatory drug (NSAID).

Preferably the active substance combination according to the present invention comprises one or more substituted carbinol compounds of general formula I given above, wherein R¹ represents a hydrogen atom, a linear or branched C₁₋₄ alkyl radical, a linear or branched C₂₋₄ alkenyl radical, a 5- or 6-membered cycloaliphatic radical, which may contain at least one nitrogen atom as ring member and/or which may be at least mono-substituted by a linear or branched C₁₋₄ alkyl radical, or a phenyl radical, preferably a hydrogen atom, a linear or branched C₁₋₄ alkyl radical, a vinyl group, a cyclohexyl radical, an N-Methyl-piperidyl radical or a phenyl radical, and the other substituents R²—R⁶, X and Y have the meaning given above, optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate.

Also preferred the active substance combination according to the present invention comprises one or more substituted carbinol compounds of general formula I given above, wherein R² represents a hydrogen atom, an optionally at least one nitrogen atom as ring member containing, 5- or 6-membered cycloaliphatic radical, which may be at least mono-substituted by a linear or branched C₁₋₄-alkyl radical and/or which may be bound vi a linear or branched C₁₋₄-alkyl radical, a NR³R⁴-moiety, which is bound via a linear or branched C₁₋₄ alkylene group, or a NR⁵R⁶-moiety, which is bound via a linear or branched C₁₋₄ alkylene group, prferably a hydrogen atom, an optionally at least one nitrogen atom as ring member containing, 5- or 6-membered cycloaliphatic radical, which may be at least mono-substituted by a linear or branched C₁₋₄-alkyl radical and/or which may be bound vi a linear or branched C₁₋₄-alkyl radical, a NR³R⁴-moiety, which is bound via a linear or branched C₁₋₄ alkylene group, or a NR⁵R⁶-moiety, which is bound via a linear or branched C₁₋₄ alkylene group, and the remaining substituents R¹, R³—R⁶, X and Y have the meaning given above, optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate.

In another preferred embodiment of the present invention the inventive active substance combination comprises one or more substituted carbinol compounds of general formula I given above, wherein R³ and R⁴, identical or different, independently from one another represent a linear or branched C₁₋₄ alkyl radical or an unsubstituted benzyl radical, preferably a linear or branched C₁₋₄ alkyl radical, and the remaining substituents R¹, R², R⁵, R⁶, X and Y have the meaning given above, optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate.

Also preferred the active substance combination according to the present invention comprises one or more substituted carbinol compounds of general formula I given above, wherein R⁵ and R⁶ together with the bridging nitrogen atom represent a saturated, unsubstituted, optionally at least one oxygen atom as ring member containing, 5- or 6-membered heterocyclic radical, and the remaining substituents R¹—R⁴, X and Y have the meaning given above, optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate.

Also preferred the active substance combination according to the present invention comprises one or more substituted carbinol compounds of general formula I given above, wherein X represents an optionally at least mono-substituted phenyl radical or an optionally at least mono-substituted thienyl radical, wherein in each case the substituents are independently selected from the group consisting of a linear or branched C₁₋₄ alkyl radical, a linear or branched C₁₋₄ alkoxy radical, a linear or branched C₁₋₄ alkyl radical, which is at least partially fluorinated, a fluorine atom, a chlorine atom and a bromine atom, preferably an optionally at least mono-substituted phenyl radical or an optionally at least mono-substituted thienyl radical, wherein in each case the substituents are independently selected from the group consisting of a methyl radical, a methoxy radical, a trifluoromethyl radical, a fluorine atom, a chlorine atom and a bromine atom, and the remaining substituents R¹—R⁶ and Y have the meaning given above, optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate.

Also preferred the active substance combination according to the present invention comprises one or more substituted carbinol compounds of general formula I, wherein Y represents an azole radical selected from the group consisting of
a) a pyrazole of the general formula (a): 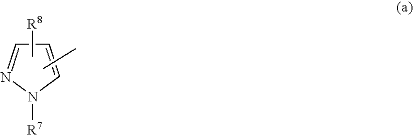
   in which R⁷ represents a linear or branched C₁₋₁₂ alkyl radical, a benzyl radical or a radical of the type: 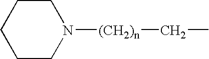
   in which n=1 or 2, and

R⁸ represents a hydrogen atom, a methyl radical or a halogen atom, preferably a hydrogen atom, a methyl radical, a bromine atom or a chlorine atom,
b) an imidazole of the general formula 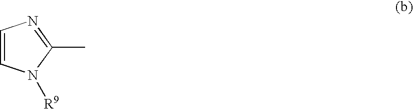
   in which R⁹ represents a hydrogen atom, a C₁₋₁₂ alkyl radical, a benzyl radical or a radical of the general formula (b1):
   R¹⁰—(CH₂)ₙ— (b1)
   in which n is 2, 3 or 4 and R¹⁰ represents a piperidinyl radical, a phenyl radical, a cyano group, a hydroxyl radical, a carboxy radical, an amino group, a dimethylamino group or a methyl ester group,
   and
   an imidazole of the following formula: 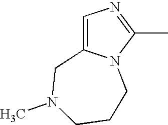
   and the remaining substituents R¹—R⁶ and X have the meaning given above, optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate.

In a particularly preferred embodiment of the present invention the inventive active substance combination comprises one or more substituted carbinol compounds of general formula I 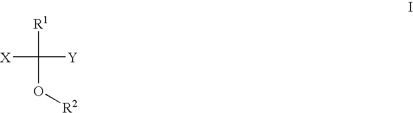
wherein
R¹ represents a hydrogen atom, a methyl radical, an ethyl radical, an n-propyl radical, an iso-propyl radical, a sec-butyl radical, a tert-butyl radical, an n-butyl radical, a vinyl radical, a cyclohexyl radical, an N-methyl-piperidinyl group, or a phenyl group,
R² represents a hydrogen atom, a dimethylaminoethyl group, a pyrrolidinylethyl group, a piperidinylethyl group, a methyl-benzyl-aminoethyl group, a morpholinylethyl group, a diisopropylaminoethyl group, a dimethylaminopropyl group, a piperidinylpropyl group, a pyrrolidinylpropyl group, a morpholinylpropyl group, an N-methyl-2-piperidyl group, an N-ethyl-2-piperidyl group, an N-propyl-2-piperidyl group, an N-methyl-2-pyrrolidinyl group, an N-ethyl-2-pyrrolidinyl group, an N-propyl-2-pyrrolidinyl group, or a 2-dimethylaminoethyl-1-methyl group,
X represents a phenyl radical, a 2-methyl-phenyl radical, a 3-methyl-phenyl radical, a 4-methyl phenyl radical, a 2-chloro-phenyl radical, a 3-chloro-phenyl radical, a 4-chloro-phenyl radical, a 2-fluoro-phenyl radical, a 3-fluoro-phenyl radical, a 4-fluoro-phenyl radical, a 2-trifluoromethyl-phenyl radical, a 3-trifluoromethyl-phenyl radical, a 4-trifluoromethyl-phenyl radical, a 2-methoxy-phenyl radical, a 3-methoxy-phenyl radical, a 4-methoxy-phenyl radical, a 3,4,5-tris-methoxy-phenyl radical, a 3,4-dichloro-phenyl radical, a 2,4-dichloro-phenyl radical, a thien-2-yl radical, a thien-3-yl radical, a 3-methyl-thien-2-yl radical, a 5-methyl-thien-2-yl-radical, a 5-bromo-thien-2-yl radical or a 4-bromo-thien-2-yl-radical,
Y represents an azole radical selected from the group consisting of
   a) a pyrazole of the general formula (a): 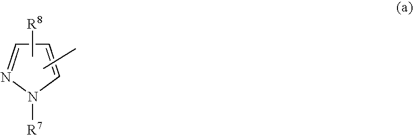
      in which
R⁷ represents a methyl radical, an ethyl radical, an n-propyl radical, an iso-propyl radical, an n-butyl radical, a sec-butyl radical or a tert-butyl radical,
R⁸ represents a hydrogen atom, a methyl radical, a bromine atom or a chlorine atom,
   b) an imidazole of the general formula 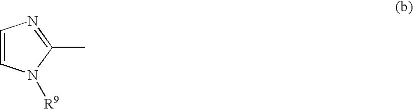
      in which R⁹ represents a hydrogen atom, a methyl radical, an ethyl radical, an n-propyl radical, an iso-butyl radical, an n-butyl radical, a sec-butyl radical a tert-butyl radical, an n-pentyl radical, an n-hexyl radical, an n-heptyl radical, an n-octyl radical, an n-nonyl radical, an n-decyl radical, an n-undecyl radical an n-dodecyl radical, a benzyl radical, or a radical of the general formula (b1):
      R¹⁰—(CH₂)ₙ— (b1)
      in which n is 2, 3 or 4 and R¹⁰ represents a piperidinyl radical, a phenyl radical, a cyano group, a hydroxyl radical, a carboxy radical, an amino group, a dimethylamino group, or a methyl ester group,
      and
   (c) an imidazole of the following formula:

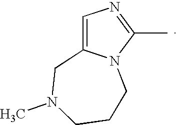

In a most particularly preferred embodiment of the present invention the inventive active substance combination comprises one or more substituted carbinol compounds selected from the group consisting of:
2-{α-[2-(dimethylamino)ethoxy]benzyl}-1-methyl-1H-imidazole,
2-{4-chloro-α-[2-(dimethylamino)ethoxy]-α-methylbenzyl}-1-methyl-1H-imidazole,
2-{4-chloro-α-[2-(dimethylamino)ethoxy]benzyl}-1-methyl-1H-imidazole,
2-{3-chloro-α-[2-(dimethylamino)ethoxy]benzyl}-1-methyl-1H-imidazole
2-{4-chloro-α-[2-(dimethylamino)ethoxy]-α-methylbenzyl}-1-methyl-1H-imidazole,
2-{4-fluoro-α-[2-(dimethylamino)ethoxy]-α-methylbenzyl}-1-methyl-1H-imidazole,
2-{α-[2-(dimethylamino)ethoxy]-α-methyl-3-(trifluoromethyl)benzyl}-1-methyl-1H-imidazole,
2-{3-chloro-α-[2-(dimethylamino)ethoxy]-α-methylbenzyl}-1-methyl-1H-imidazole,
2-{3-chloro-α-[2-(dimethylamino)ethoxy]-α-propylbenzyl}-1-methyl-1H-imidazole,
1-butyl-2-{4-chloro-α-[2-(dimethylamino)ethoxy]-α-methylbenzyl}-1H-imidazole,
2-{α-[2-(dimethylamino)ethoxy]-α-methyl-4-methoxybenzyl}-1-methyl-1H-imidazole,
2-{3-chloro-α-methyl-α-[2-(N-pyrrolidinyl)ethoxy]benzyl}-1-methyl-1H-imidazole,
2-{α-[2-(dimethylamino)ethoxy]-α-propyl-3,4,5-trimethoxybenzyl}-1-dodecyl-1H-imidazole,
1-butyl-2-{α-[2-(dimethylamino)ethoxy]-4-(trifluoromethyl)benzyl}-1H-imidazole,
1-methyl-2-{α-methyl-α-[2-(N-piperidyl)ethoxy]-3-(trifluoromethyl)benzyl}-1H-imidazole,
2-{α-cyclohexyl-3,4-dichloro-α-[2-(dimethylamino)ethoxy]benzyl}-1-methyl-1H-imidazole,
2-{3,4-dichloro-α-[2-(dimethylamino)ethoxy]-α-propylbenzyl}-1-methyl-1H-imidazole,
2-{3,4-dichloro-α-[2-(dimethylamino)ethoxy]-α-methylbenzyl}-1-methyl-1H-imidazole,
2-{3,4-dichloro-α-[2-(dimethylamino)ethoxy]benzyl}-1-methyl-1H-imidazole,
2-{4-chloro-α-[2-(dimethylamino)ethoxy]-α-methylbenzyl}-1-[2-(N-piperidyl)ethyl]-1H-imidazole,
2-{4-chloro-α-[2-(dimethylamino)ethoxy]-α-methylbenzyl}-1-[2-(N-piperidyl)propyl]-1H-imidazole,
1-(3-cyanopropyl)-2-{4-chloro-α-[2-(dimethylamino)ethoxy]benzyl}-1H-imidazole,
2-{4-chloro-α-[2-(dimethylamino)ethoxy]-α-(N-methyl-4-piperidyl)benzyl}-1-methyl-1H-imidazole,
1-benzyl-2-{α-[2-(N-benzyl-N-methylamino)ethoxy]-4-chlorobenzyl}-1H-imidazole,
2-{4-chloro-α-[2-(dimethylamino)ethoxy]-α-methylbenzyl}-7-methyl-6,7,8,9-tetrahydro-1H-imidazole[1,5-a][1,4]diazepine,
2-{4-chloro-α-[2-(dimethylamino)ethoxy]benzyl}-7-methyl-6,7,8,9-tetrahydro-1H-imidazole[1,5-a][1,4]diazepine,
1-butyl-5-{α-[2-(dimethylamino)ethoxy]benzyl}-1H-pyrazole,
5-{α-(4-chlorophenyl)-α-[2-(dimethylamino)ethoxy]benzyl}-1-methyl-1H-pyrazole,
1-butyl-5-{α-[2-(dimethylamino)ethoxy]-3,4,5-trimethoxybenzyl}-1H-pyrazole,
1-butyl-5-{4-chloro-α-[2-(dimethylamino)ethoxy]-α-methylbenzyl}-1H-pyrazole,
5-{α-[2-(dimethylamino)ethoxy]benzyl}-1-methyl-1H-pyrazole,
5-{α-[2-(dimethylamino)ethoxy]-α-methylbenzyl}-1-methyl-1H-pyrazole,
5-{α-[2-(dimethylamino)ethoxy]-3,4,5-trimethoxybenzyl}-1-methyl-1H-pyrazole,
1-methyl-5-{α-[2-(N-pyrrolidinyl)ethoxy]benzyl}-1H-pyrazole,
1-methyl-5-{α-[2-(N-morpholinyl)ethoxy]benzyl}-1H-pyrazole,
5-{α-[2-(dimethylamino)ethoxy]-α-methyl-3,4,5-trimethoxybenzyl}-1-methyl-1H-pyrazole,
4-bromo-5-{α-[2-(dimethylamino)ethoxy]benzyl}-1-methyl-1H-pyrazole,
1,3-dimethyl-5-{α-[2-(dimethylamino)ethoxy]-α-methylbenzyl}-1H-pyrazole,
1,3-dimethyl-5-{α-[2-(dimethylamino)ethoxy]benzyl}-1H-pyrazole,
5-{α-[2-(dimethylamino)ethoxy]-2-methylbenzyl}-1-methyl-1H-pyrazole,
4-chloro-5-{4-chloro-α-[2-(dimethylamino)ethoxy]benzyl}-1-methyl-1H-pyrazole,
5-{4-chloro-α-[2-(dimethylamino)ethoxy]benzyl}-1-methyl-1H-pyrazole,
5-{3-chloro-α-[2-(dimethylamino)ethoxy]benzyl}-1-methyl-1H-pyrazole,
5-{α-[2-(dimethylamino)ethoxy]-4-methylbenzyl}-1-methyl-1H-pyrazole,
5-{2-chloro-α-[2-(dimethylamino)ethoxy]benzyl}-1-methyl-1H-pyrazole,
1-methyl-5-{α-[2-(N-piperidyl)ethoxy]benzyl}-1H-pyrazole,
1-methyl-5-{α-[2-(N-propyl-2-piperidyl)ethoxy]benzyl}-1H-pyrazole,
5-{α-[2-(N-ethyl-2-piperidyl)ethoxy]benzyl}-1-methyl-1H-pyrazole,
1-methyl-5-{α-[2-(N-methyl-2-pyrrolidinyl)ethoxy]benzyl}-1H-pyrazole,
5-{α-[2-(diisopropylamino)ethoxy]benzyl}-1-methyl-1H-pyrazole,
1-methyl-5-{α-[2-(N-methyl-2-piperidyl)ethoxy]benzyl}-1H-pyrazole,
2-{4-chloro-α-[3-(dimethylamino)propoxy]-α-methylbenzyl}-1-methyl-1H-imidazole,
2-{3-chloro-α-[3-(dimethylamino)propoxy]benzyl}-1-methyl-1H-imidazole
2-{4-chloro-α-[3-(dimethylamino)propoxy]-α-ethylbenzyl}-1-methyl-1H-imidazole,
2-{α-butyl-3-chloro-α-[3-(dimethylamino)propoxy]benzyl}-1-methyl-1H-imidazole,
2-{α-cyclohexyl-4-chloro-α-[3-(dimethylamino)propoxy]benzyl}-1-methyl-1H-imidazole,
2-{α-[3-(dimethylamino)propoxy]-4-fluoro-α-methylbenzyl}-1-methyl-1H-imidazole,
2-{α-[3-(dimethylamino)propoxy]-α-methyl-3-(trifluoromethyl)benzyl}-1-methyl-1H-imidazole,
2-{2-chloro-α-[3-(dimethylamino)propoxy]-α-methylbenzyl}-1-methyl-1H-imidazole,
2-{3-chloro-α-[3-(dimethylamino)propoxy]-α-methylbenzyl}-1-methyl-1H-imidazole,
2-{α-[3-(dimethylamino)propoxy]-α-methyl-3,4,5-trimethoxybenzyl}-1-methyl-1H-imidazole,
2-{α-[3-(dimethylamino)propoxy]-α-methyl-4-methoxybenzyl}-1-methyl-1H-imidazole,
2-{4-chloro-α-[3-(dimethylamino)propoxy]benzyl}-1-methyl-1H-imidazole,
2-{α-[3-(dimethylamino)propoxy]-3,4,5-trimethoxybenzyl)-1-methyl-1H-imidazole,
2-{α-[3-(dimethylamino)propoxy]-α-methyl-4-(trifluoromethyl)benzyl}-1-methyl-1H-imidazole,
2-{α-[3-(dimethylamino)propoxy]-3-(trifluoromethyl)benzyl}-1-methyl-1H-imidazole,
2-{α-[3-(dimethylamino)propoxy]-4-(trifluoromethyl)benzyl}-1-methyl-1H-imidazole,
2-{α-[3-(dimethylamino)propoxy]-4-methoxybenzyl}-1-methyl-1H-imidazole,
2-{α-butyl-α-[3-(dimethylamino)propoxy]-3-(trifluoromethyl)benzyl}-1-methyl-1H-imidazole,
1-butyl-2-{4-chloro-α-[3-(dimethylamino)propoxy]-α-methylbenzyl}-1H-imidazole,
1-butyl-2-{α-butyl-α-[3-(dimethylamino)propoxy]-3,4,5-trimethoxybenzyl}-1H-imidazole,
1-butyl-2-{α-butyl-2-chloro-α-[3-(dimethylamino)propoxy]benzyl}-1H-imidazole,
1-butyl-2-{α-butyl-2,4-dichloro-α-[3-(dimethylamino)propoxy]benzyl}-1H-imidazole,
1-butyl-2-{α-[3-(dimethylamino)propoxy]-4-(trifluoromethyl)benzyl}-1H-imidazole,
2-{4-chloro-α-[3-(N-piperidyl)propoxy]benzyl}-1-methyl-1H-imidazole,
1-methyl-2-{α-methyl-α-[3-(N-piperidyl)propoxy]-4-(trifluoromethyl)benzyl}-1H-imidazole,
2-{α-butyl-2-chloro-α-[3-(dimethylamino)propoxy]benzyl}-1-methyl-1H-imidazole,
2-{α-butyl-3,4-dichloro-α-[3-(dimethylamino)propoxy]benzyl}-1-methyl-1H-imidazole,
2-{3,4-dichloro-α-[3-(dimethylamino)propoxy]-α-methylbenzyl}-1-methyl-1H-imidazole,
2-{3,4-dichloro-α-[3-(dimethylamino)propoxy]benzyl}-1-methyl-1H-imidazole,
2-{α-cyclohexyl-3,4-dichloro-α-[3-(dimethylamino)propoxy]benzyl}-1-methyl-1H-imidazole,
2-{4-chloro-α-[3-(dimethylamino)propoxy]-α-methylbenzyl}-α-[2-(N-piperidyl)ethyl]-1H-imidazole,
2-{4-chloro-α-[3-(dimethylamino)propoxy]-α-methylbenzyl}-1-[2-(N-piperidyl)propyl]-1H-imidazole,
2-{4-chloro-α-[3-(dimethylamino)propoxy]-α-(N-methyl-4-piperidyl)benzyl}-1-methyl-1H-imidazole,
1-butyl-5-{α-[3-(dimethylamino)propoxy]benzyl}-1H-pyrazole,
1-butyl-5-{4-chloro-α-[3-(dimethylamino)propoxy]-α-methylbenzyl}-1H-pyrazole,
5-{α-[3-(dimethylamino)propoxy]benzyl}-1-methyl-1H-pyrazole,
5-{α-[3-(dimethylamino)propoxy]-α-methylbenzyl}-1-methyl-1H-pyrazole,
1,3-dimethyl-5-{α-[3-(dimethylamino)propoxy]-α-methylbenzyl}-1H-pyrazole,
1,3-dimethyl-5-{α-[3-(dimethylamino)propoxy]benzyl}-1H-pyrazole,
5-{α-[3-(dimethylamino)propoxy]-2-methylbenzyl}-1-methyl-1H-pyrazole,
5-chloro-5-{4-chloro-α-[3-(dimethylamino)propoxy]benzyl}-1-methyl-1H-pyrazole,
1-methyl-5-{α-[3-(N-piperidyl)propoxy]benzyl}-1H-pyrazole,
1-methyl-5-{α-[3-(N-pyrrolidinyl)propoxy]benzyl}-1H-pyrazole,
4-{4-chloro-α-[2-(dimethylamino)ethoxy]benzyl}-1-methyl-1H-pyrazole,
4-{4-chloro-α-[2-(dimethylamino)ethoxy]-α-methylbenzyl}-1-methyl-1H-pyrazole,
4-{4-chloro-α-[2-(N-propyl-2-piperidyl)ethoxy]benzyl)-1-methyl-1H-pyrazole,
4-{4-chloro-α-[2-(N-methyl-2-piperidyl)ethoxy]benzyl}-1-methyl-1H-pyrazole,
4-{4-chloro-α-[2-(N-ethyl-2-piperidyl)ethoxy]benzyl}-1-methyl-1H-pyrazole,
4-{4-chloro-α-[2-(diisopropylamino)ethoxy]benzyl}-1-methyl-1H-pyrazole,
4-{4-chloro-α-[2-(N-methyl-2-pyrrolidinyl)ethoxy]benzyl}-1-methyl-1H-pyrazol
4-{α-[3-(dimethylamino)propoxy]benzyl}-1-methyl-1H-pyrazole,
4-{4-chloro-α-[3-(N-morpholinyl)propoxy]benzyl}-1-methyl-1H-pyrazole,
4-{4-chloro-α-[3-(N-pyrrolidinyl)propoxy]benzyl}-1-methyl-1H-pyrazole,
2-(α-hydroxybenzyl)-1H-imidazole,
2-(4-chloro-α-hydroxybenzyl)-1H-imidazole,
2-(4-chloro-α-hydroxybenzyl)-1-methyl-1H-imidazole,
2-(3-chloro-α-hydroxybenzyl)-1-methyl-1H-imidazole,
2-(4-fluoro-α-hydroxybenzyl)-1-methyl-1H-imidazole,
2-[α-hydroxy-3-(trifluoromethyl)benzyl]-1-methyl-1H-imidazole,
2-[α-hydroxy-4-(trifluoromethyl)benzyl]-1-methyl-1H-imidazole,
2-(α-hydroxy-3,4,5-trimethoxybenzyl)-1-methyl-1H-imidazole,
2-(3,4-dichloro-α-hydroxybenzyl)-1-methyl-1H-imidazole,
1-butyl-2-[α-hydroxy-4-(trifluoromethyl)benzyl]-1H-imidazole,
1-butyl-2-(3,4-dichloro-α-hydroxybenzyl)-1H-imidazole,
1-butyl-2-(4-chloro-α-hydroxybenzyl)-1H-imidazole,
1-butyl-2-(α-hydroxy-3,4,5-trimethoxybenzyl)-1H-imidazole,
1-dodecyl-2-(α-hydroxy-3,4,5-trimethoxybenzyl)-1H-imidazole,
2-(α-butyl-3-chloro-α-hydroxybenzyl)-1-methyl-1H-imidazole,
2-(3-chloro-α-hydroxy-α-methylbenzyl)-1-methyl-1H-imidazole,
2-(4-chloro-α-hydroxy-α-methylbenzyl)-1-methyl-1H-imidazole,
2-[4-chloro-α-hydroxy-α-(N-methyl-4-piperidyl)benzyl]-1-methyl-1H-imidazole,
2-(4-chloro-α-ethyl-α-hydroxybenzyl)-1-methyl-1H-imidazole,
2-(α-butyl-4-chloro-α-hydroxybenzyl)-1-methyl-1H-imidazole,
2-(α-cyclohexyl-4-chloro-α-hydroxybenzyl)-1-methyl-1H-imidazole,
2-(2-chloro-α-hydroxy-α-methylbenzyl)-1-methyl-1H-imidazole,
2-(α-butyl-2-chloro-α-hydroxybenzyl)-1-methyl-1H-imidazole,
2-[α-hydroxy-α-methyl-3-(trifluoromethyl)benzyl]-1-methyl-1H-imidazole,
2-[α-butyl-α-hydroxy-3-(trifluoromethyl)benzyl]-1-methyl-1H-imidazole
2-[α-cyclohexyl-α-hydroxy-3-(trifluoromethyl)benzyl]-1-methyl-1H-imidazole,
2-[α-hydroxy-α-methyl-4-(trifluoromethyl)benzyl]-1-methyl-1H-imidazole,
2-(4-fluoro-α-hydroxy-α-methylbenzyl)-1-methyl-1H-imidazole,
2-(α-hydroxy-α-methyl-4-methoxybenzyl)-1-methyl-1H-imidazole,
2-(3,4-dichloro-α-hydroxy-α-methylbenzyl)-1-methyl-1H-imidazole,
2-(α-butyl-3,4-dichloro-α-hydroxybenzyl)-1-methyl-1H-imidazole,
2-(α-cyclohexyl-3,4-dichloro-α-hydroxybenzyl)-1-methyl-1H-imidazole,
2-(α-hydroxy-α-methyl-3,4,5-trimethoxybenzyl)-1-methyl-1H-imidazole,
1-butyl-2-(4-chloro-α-hydroxy-α-methylbenzyl)-1H-imidazole,
1-butyl-2-(α-butyl-4-chloro-α-hydroxybenzyl]-1H-imidazole,
1-butyl-2-[4-chloro-α-hydroxy-α-(N-methyl-4-piperidyl)benzyl]-1H-imidazole,
1-butyl-2-(α-butyl-α-hydroxy-3,4,5-trimethoxybenzyl)-1H-imidazole,
1-butyl-2-(α-butyl-2-chloro-α-hydroxybenzyl)-1H-imidazole,
1-butyl-2-[α-ethyl-α-hydroxy-3-(trifluoromethyl)benzyl]-1H-imidazole,
1-butyl-2-(α-butyl-2,4-dichloro-α-hydroxybenzyl)-1H-imidazole,
2-(4-chloro-α-hydroxy-α-methylbenzyl)-1-[2-(N-piperidyl)ethyl]-1H-imidazole,
2-(4-chloro-α-hydroxy-α-methylbenzyl)-1-(3-dimethylaminopropyl)-1H-imidazole,
2-(α-butyl-α-hydroxy-3,4,5-trimethoxybenzyl)-1-dodecyl-1H-imidazole,
1-benzyl-2-[α-butyl-α-hydroxy-3-(trifluoromethyl)benzyl]-1H-imidazole,
1-benzyl-2-(4-chloro-α-hydroxy-α-methylbenzyl)-1H-imidazole,
1-(2-cyanoethyl)-2-(4-chloro-α-hydroxybenzyl)-1H-imidazole,
1-(3-aminopropyl)-2-(4-chloro-α-hydroxybenzyl)-1H-imidazole,
3-[2-(3-chloro-α-hydroxybenzyl)-1H-imidazole-1-yl]propanoic acid
2-(4-chloro-α-hydroxybenzyl)-1-(3-hydroxypropyl)-1H-imidazole,
3-[2-(3-chloro-α-hydroxybenzyl)-1H-imidazole-1-yl]methyl-propanoate
2-(α-hydroxybenzyl)-1-(3-hydroxypropyl)-1H-imidazole,
2-(α-hydroxy-4-methylbenzyl)-1-(3-hydroxypropyl)-1H-imidazole,
2-(α-hydroxy-4-methoxybenzyl)-1-(3-hydroxypropyl)-1H-imidazole,
2-(3,4-dichloro-α-hydroxybenzyl)-1-(3-hydroxypropyl)-1H-imidazole,
3-{2-(α-hydroxybenzyl)-1H-imidazole-1-yll}-methyl propanoate
2-(4-chloro-α-hydroxybenzyl)-1-(4-hydroxybutyl)-1H-imidazole,
1-(3-cyanopropyl)-2-(4-chloro-α-hydroxybenzyl)-1H-imidazole,
4-[2-(4-chloro-α-hydroxybenzyl)-1H-imidazole-1-yl]butanoic acid,
4-[2-(4-chloro-α-hydroxybenzyl)-1H-imidazole-1-yl]-methyl butanoate,
1-butyl-5-(α-hydroxybenzyl)-1H-pyrazole,
5-(4-chloro-α-hydroxybenzyl)-1-methyl-1H-pyrazole,
5-(α-hydroxy-3,4,5-trimethoxybenzyl)-1-methyl-1H-pyrazole,
1-butyl-5-(α-hydroxy-3,4,5-trimethoxybenzyl)-1H-pyrazole,
4-bromo-5-(α-hydroxybenzyl)-1-methyl-1H-pyrazole,
5-[α-(4-chlorophenyl)-α-hydroxybenzyl]-1-methyl-1H-pyrazole,
1-butyl-5-(4-chloro-α-hydroxy-α-methylbenzyl)-1H-pyrazole,
5-(α-hydroxy-α-methylbenzyl)-1-methyl-1H-pyrazole,
5-(α-hydroxy-α-methyl-3,4,5-trimethoxybenzyl)-1-methyl-1H-pyrazole,
1,3-dimethyl-5-(α-hydroxy-α-methylbenzyl)-1H-pyrazole,
1-butyl-5-(α-hydroxy-α-vinylbenzyl)-1H-pyrazole,
1-butyl-5-(4-chloro-α-hydroxy-α-vinylbenzyl)-1H-pyrazole,
4-chloro-5-(α-hydroxybenzyl)-1-methyl-1H-pyrazole,
5-(α-hydroxy-2-methylbenzyl)-1-methyl-1H-pyrazole,
5-(3-chloro-α-hydroxybenzyl)-1-methyl-1H-pyrazole,
5-(α-hydroxy-4-methylbenzyl)-1-methyl-1H-pyrazole,
5-(2-chloro-α-hydroxybenzyl)-1-methyl-1H-pyrazole,
5-(α-hydroxy-4-methoxybenzyl)-1-methyl-1H-pyrazole,
5-{(1-[2-(dimethylamino)ethoxy]-2-thienylmethyl}-1-methyl-1H-pyrazole,
5-{α-[2-(dimethylamino)ethoxy]-2-thienylmethyl}-1-methyl-1H-pyrazole citrate,
5-{α-[2-(dimethylamino)ethoxy]-3-thienylmethyl}-1-methyl-1H-pyrazole,
2-{α-[2-(dimethylamino)ethoxy]-2-thienylmethyl}-1-methyl-1H-imidazole,
5-{α-[2-(dimethylamino)ethoxy]-3-methyl-2-thienylmethyl}-1-methyl-1H-pyrazole,
5-{α-[2-(dimethylamino)ethoxy]-5-methyl-2-thienylmethyl}-1-methyl-1H-pyrazole,
5-{5-bromo-α-[2-(dimethylamino)ethoxy]-2-thienylmethyl}-1-methyl-1H-pyrazole,
5-{4-bromo-α-[2-(dimethylamino)ethoxy]-2-thienylmethyl}-1-methyl-1H-pyrazole,
5-{α-[2-(dimethylamino)ethoxy]-α-methyl-2-thienylmethyl}-1-methyl-1H-pyrazole,
5-{α-[2-(dimethylamino)ethoxy]benzyl}-1-methyl-1H-pyrazole citrate
(±)-5-{α-[2-(dimethylamino)-1-(methyl)ethoxy]benzyl}-1-methyl-1H-pyrazole,
(±)-5-{α-[2-(dimethylamino)-1-(methyl)ethoxy]benzyl}-1-methyl-1H-pyrazole,
(+)-5-{α-[2-(dimethylamino)ethoxy]-2-thienylmethyl}-1-methyl-1H-pyrazole,
(−)-5-{α-[2-(dimethylamino)ethoxy]-2-thienylmethyl}-1-methyl-1H-pyrazole,
(+)-5-{α-[2-(dimethylamino)ethoxy]-2-thienylmethyl}-1-methyl-1H-pyrazole citrate,
(−)-5-{α-[2-(dimethylamino)ethoxy]-2-thienylmethyl}-1-methyl-1H-pyrazole citrate,
(+)-5-{α-[2-(dimethylamino)ethoxy]-2-thienylmethyl}-1-methyl-1H-pyrazole-D-ditoluyltartrate,
(−)-5-{α-[2-(dimethylamino)ethoxy]-2-thienylmethyl}-1-methyl-1H-pyrazole D-ditoluyltartrate,
(+)-5-{α-[2-(dimethylamino)ethoxy]benzyl}-1-methyl-1H-pyrazole citrate,
(−)-5-{α-[2-(dimethylamino)ethoxy]benzyl}-1-methyl-1H-pyrazole citrate,
5-(α-hydroxy-2-thienylmethyl)-1-methyl-1H-pyrazole,
5-(α-hydroxy-3-methyl-2-thienylmethyl)-1-methyl-1H-pyrazole,
5-(α-hydroxy-5-methyl-2-thienylmethyl)-1-methyl-1H-pyrazole,
5-(5-bromo-α-hydroxy-2-thienylmethyl)-1-methyl-1H-pyrazole,
5-(4-bromo-α-hydroxy-2-thienylmethyl)-1-methyl-1H-pyrazole and
5-(α-hydroxy-α-methyl-2-thienylmethyl)-1-methyl-1H-pyrazole
   as component (A).

The preparation of the substituted carbinol compounds of general formula 1, their stereoisomers, corresponding salts and corresponding solvates may be accomplished by the reagents and methods described, for example, in EP 0 289 380, U.S. Pat. No. 5,017,596, WO99/52525 (U.S. Pat. No. 6,410,582) and WO99/07684 (U.S. Pat. No. 6,118,009). Methods for the optical resolution of said compounds, i.e. the preparation or separation of the respective stereoisomers are described, for example, in WO99/02500 (U.S. Pat. No. 6,187,930) and WO97/20817 (U.S. Pat. No. 5,849,931). The corresponding parts of these publications are hereby incorporated by reference and form part of the present disclosure.

Physiologically acceptable salts of the substituted carbinol compounds of general formula I given above may be obtained by conventional methods known to those skilled in the art. Preferred pharmaceutically acceptable salts of these substituted carbinol compounds of general formula I given above are the citrate salts or the ditoluyltartrate salts. Generally included are also addition salts of mineral acids or of organic acids such as oxalate, tartrate, citrate and hydroquinonesulfate. Additionally, the term “salt” herein is to be understood as including any form of an active compound of the inventive active substance combination in which this is present in ionic or charged form and is coupled with a corresponding counter-ion (a cation or anion) or is in solution. The term “salt” further comprises complexes of an active compound of the inventive active substance combination with other ions or molecules, in particular complexes, which are complexed via ionic interactions.

In the context of the present invention, the term “physiologically acceptable salt” is understood in particular as including a salt that is formed either with a physiologically tolerated acid, that is to say salts of the particular active compound with inorganic or organic acids which are physiologically tolerated—especially if used on humans and/or mammals—or with at least one, preferably inorganic, ion, preferably cation, which are physiologically tolerated, especially if used on humans and/or mammals. Examples of physiologically tolerated salts of particular acids are salts of hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1,2-dihydro-6-benzo[d]isothiazol-3-one (saccharin acid), monomethylsebacic acid, 5-oxo-proline, hexane-1-sulfonic acid, nicotinic acid, 2-, 3- or 4-aminobenzoic acid, 2,4,6-trimethyl-benzoic acid, alpha-lipoic acid, acetylglycine, acetylsalicylic acid, hippuric acid and/or aspartic acid. Examples of physiologically tolerated salts of particular bases are salts of alkali and alkaline earth metals and/or with {NHₓR₄₋ₓ]⁺-ions, wherein x is 0, 1, 2, 3 or 4 and R represents a linear or branched C₁₋₄ alkyl radical.

With regard to the compounds of component A of the inventive active substance combination the salts that are preferred are salts of physiologically tolerated acids.

The salt, which is particularly preferred for the particular compound of component A is the citrate.

Non-steroidal anti-inflammatory drugs according to component (B) of the present invention include corresponding salts and corresponding solvates of these drugs as well. Physiologically acceptable salts and solvates of these compounds of component (B) may be obtained by conventional methods known to those skilled in the art

Suitable non-steroidal anti-inflammatory drugs (NSAIDS) according to component (B) of the inventive active substance combination, suitable doses for the administration to patients as well as methods for their preparation are well known to those skilled in the art, e.g. from E. Friderichs, T. Christoph and H. Buschmann, “Analgesics and Antipyretics”, Ullmann's Encyclopedia of Industrial Chemistry, Sixth Edition, Wiley-VCH Verlag GmbH, Weinheim, Germany 2000, pages 3-24 and H. Buschmann, T. Christoph, E. Friderichs, C. Maul, B. Sundermann (Editiors), “Analgesics—From Chemistry and Pharmacology to Clinical Application”, 1. Edition 2002-Part II-pages 13-126 Wiley-VCH Verlag, Weinheim, Germany. The respective parts of the description are hereby incorporated by reference and form part of the present disclosure.

Preferably the active substance combination of the present invention comprises compounds with Cyclooxygenase-1 and/or Cyclooxygenase-2 inhibiting activity selected from the group consisting of Acemetacin, Acetylsalicylic acid, Bufexamac, Diclofenac, Diflunisal, Ethenzamide, Etofenamate, Fenbufen, Fenoprofen, Feprazone, Flobufen, Flufenamic acid, Flurbiprofen, Ibuprofen, Indomethacin, Isoxicam, Kebuzone, Ketoprofen, Ketorolac, Lonazolac, Lornoxicam, Meclofenamic acid, Mefenamic acid, Metamizol, Mofebutazone, Nabumetone, Naproxen, Niflumic acid, Oxaprozin, Oxyphenbutazone, Paracetamol, Phenidine, Phenylbutazone, Piroxicam, Propacetamol, Propyphenazone, Salicylamide, Sulindac, Tenoxicam, Tiaprofenic acid, Tolmetin, Celecoxib, Etodolac, Etoricoxib, Meloxicam, Nimesulide, Parecoxib, Rofecoxib, Valdecoxib and physiologically acceptable salts thereof.

More preferably the pharmacologically active substance combination of the present invention comprises as component (B) one or more non-steroidal anti-inflammatory drugs selected from the group of compounds showing Cyclooxygenase-1 specific inhibition or balanced Cyclooxygenase-1 and Cyclooxygenase-2 inhibition—typically referred to as Cyclooxygenase-1 inhibitors by those skilled in the art—and Cyclooxygenase-2 Inhibitors of the first generation.

Preferably such Cyclooxygenase-1-inhibitors may be selected from the group consisting of Acemetacin, Acetylsalicylic acid, Bufexamac, Diclofenac, Diflunisal, Ethenzamide, Etofenamate, Fenbufen, Fenoprofen, Feprazone, Flobufen, Flufenamic acid, Flurbiprofen, Ibuprofen, Indomethacin, Isoxicam, Kebuzone, Ketoprofen, Ketorolac, Lonazolac, Lornoxicam, Meclofenamic acid, Mefenamic acid, Metamizol, Mofebutazone, Nabumetone, Naproxen, Niflumic acid, Oxaprozin, Oxyphenbutazone, Paracetamol, Phenidine, Phenylbutazone, Piroxicam, Propacetamol, Propyphenazone, Salicylamide, Sulindac, Tenoxicam, Tiaprofenic acid, Tolmetin and physiologically acceptable salts thereof.

For the purpose of the present invention Cyclooxygenase-2 Inhibitors of the first generation are compounds having a less or equal to 100-fold selectivity for Cyclooxygenase-2 compared to Cyclooxygenase-1, whereby said selectivity is determined according to the method described in L. Cullen et al., JPET, Vol. 287, 578-582, 1998 and A. Hiermann et al., Inflamm. Res., Vol. 47, 421-427, 1998. The respective descriptions are hereby incorporated by reference and form part of the present disclosure.

Suitable Cyclooxygenase-2 inhibitors of the first generation may preferably be selected from the group consisting of Etodolac, Meloxicam, Nimesulide and physiologically acceptable salts thereof.

Particularly preferably the active substance combination of the present invention comprises as component (B) one or more nonsteroidal anti-inflammatory drugs selected from the group of Cyclooxygenase-1 inhibitors, whereby those Cyclooxygenase-1 inhibitors mentioned above may preferably be present. Particularly preferably the active substance combinaton comprises as component (b) one or more Cyclooxygenase-1 inhibitors selected from the group consisting of Acetylsalicylic acid, Diclofenac, Ibuprofen, Naproxen and physiologically acceptable salts thereof.

The molar ratio between the components of the active substance combination may vary over a broad range. Preferably the molar ratio of component (A) to component (B) in the active substance combination of the present invention is in the range of 1:10to 10:1, more preferably from 1:4to4:1.

Many of the non-steroidal antiinflammatory drugs according to component (B) of the inventive active substance combination are known to exist in the form of physiologically acceptable salts, particularly those having one or more acid groups. Preferably such physiologically acceptable salts of these compounds may be selected from the group consisting of alkali metal salts, preferably potassium or sodium salts, and earth metal salts. The compounds of component (B) as well as the compounds of component (A) may each be present in form of mixture of two or more different salts.

Many of the carbinol compounds of component (A) as well as many NSAIDs of component (B) may occur in form of a corresponding ether, ester or other derivative thereof. All of these compounds are also included by the present invention. Such suitable ethers, esters and other derivatives of the compounds of components (A) and (B) as well as methods for their preparation are well known to those skilled in the art.

If the active compound of component (A) comprises at least one basic group and the active compound of component (B) comprises at least one acidic group or vice versa, both components may at least partially form a salt with one another. The salts may be prepared, optionally purified and/or optionally isolated according to conventional methods well known to those skilled in the art, e.g. by dissolution of both components in a suitable solvent, evaporation of the solvent and subsequent purification, e.g. via chromatographic methods. The respective salt may also be formed in-situ, i.e. during the process of formulating the active substance combination into a particular dosage form.

Thus, in another preferred embodiment of the present invention component (A) and component (B) are at least partially present in form of a salt formed between these two components.

Preferably component (A) and component (B) are present in the inventive active substance combination in form of a 1:1 salt, whereby said 1:1 salts may preferably be selected from the group consisting of
(a) R-(+)-5-[α-[2-(Dimethylamino)ethoxy]benzyl]-1-methyl-1H-pyrazole naproxenate (R-(+)-Cizolirtine naproxenate),
(b) S-(−)-5-[α-[2-(Dimethylamino)ethoxy]benzyl]-1-methyl-1H-pyrazole naproxenate (S-(−)-Cizolirtine naproxenate),
(c) R-(+)-5-[α-[2-(Dimethylamino)ethoxy]benzyl]-1-methyl-1H-pyrazole diclofenacate (R-(+)-Cizolirtine diclofenacate),
(d) S-(−)-5-[α-[2-(Dimethylamino)ethoxy]benzyl]-1-methyl-1H-pyrazole diclofenacate (R-(+)-Cizolirtine diclofenacate),
(e) R-(+)-5-[α-[2-(Dimethylamino)ethoxy]benzyl]-1-methyl-1H-pyrazole S-(+)-ibuprofenate (R-(+)-Cizolirtine S-(+)-ibuprofenate) and
(f) S-(−)-5-[α-[2-(Dimethylamino)ethoxy]benzyl]-1-methyl-1H-pyrazole S-(+) ibuprofenate (R-(+)-Cizolirtine S-(+)-ibuprofenate).

The inventive active substance combination is suitable for the administration to humans, including infants, children and grown-ups, as well as for the administration to animals.

Preferably the total amount of the compound(s) according to component (A), referred to as the free compound, to be administered to the patient in a 24 hours period does not exceed 800 mg.

The total amount of the compound(s) according to component (B), also referred to as the free compound, preferably does not exceed the daily dosis typically administered—if the respective compound were administered alone. Suitable dosis for the respective NSAIDS are well-known to those skilled in the art, e.g. from the respective publications given above.

Preferably the inventive active substance combination comprises components (A) and (B) in the above defined molar ratios and within the afore given limits for the maximum dosis to be administered per day.

Pharmaceutically active substances, particularly analgesics are sometimes the subject of abuse. For example, an overdose of such an analgesic may be used in an attempt to commit suicide.

Thus, in another preferred embodiment of the present invention the active substance combination further comprises as component (C) one or more agents that are suitable to reduce, preferably prevent abuse of the active substances of component (A) and/or component (B).

If such anti-abuse agents are present in the inventive active substance combination, they are included in such a form that they are either not liberated at all or in such a way that they do not limit their anti-abuse effect if the active substance combination is administered to the patient according to its intended route of administration. However, if the inventive active substance combination or—after separation—one of its components alone is administered via a route other than the intended route of administration, said anti-abuse agent will exert its effect and therefore reduce, preferably prevent abuse.

Suitable agents for reduction, preferably prevention of the abuse of these pharmacologically active components include aversive agents such as bittering agents, irritants, emetics, nauseants, and gelling agents, whereby two representatives of one class of these anti-abuse agents or two or more representatives of different classes of anti-abuse agents may be included in the active substance combination of the present invention to prevent, preferably to at least reduce different kinds of abuse.

Abuse of the inventive active substance combination may, for example, be reduced, preferably be prevented by the inclusion of an emetic. The amount of said emetic is chosen in such a way thay it will not exert its emetic effect if the active substance combination is taken in a dose intended for the prophylaxis and/or treatment of the respective disorder. However, if said dose will exceed a certain limit, which is considered harmful for the patient, the accumulated dose of the emetic will exert its emetic effect.

Suitable anti-abuse agents according to component (C) of the inventive substance combination, suitable amounts as well as methods for their incorporation into pharmceutical formulations are well-known to those skilled in the art, e.g. from WO03/013476 and WO 99/32120. The respective parts of the descriptions are hereby incorporated by references and form part of the present disclosure.

In another aspect the present invention relates to a medicament comprising an inventive active substance combination and optionally at least one further active substance and/or optionally at least one auxiliary.

Preferably the inventive medicament is suitable for the treatment of pain, whereby said pain is preferably selected from the group consisting of neuropathic pain, acute pain, chronic pain, post-operative pain, chronic lower back pain, cluster headaches, herpes neuralgia, phantom limb pain, central pain, dental pain, resistant pain, visceral pain, surgical pain, bone injury pain, pain during labor and delivery, pain resulting from burns, pain resulting from sunburns, post partum pains, migraine, angina pain, genitourinary tract-related pain, pain from cystitis and nociceptive pain, for the prophylaxis and/or treatment of neurogenic inflammation, for the prophylaxis and/or treatment of urinary incontinence, for the prophylaxis and/or treatment of depression, for the prophylaxis and/or treatment of inflammation and/or for the prophylaxis and/or treatment of inflammation related disorders, whereby said inflammation-related disorders may preferably be selected from the group consisting of arthritis, rheumatoid arthritis, spondyloarthropathies, gouty arthritis, osteoarthritis, systemic lupus erythematosus, juvenile arthritis, rheumatic fever, symptoms associated with influenza or other viral infections, common cold, lower back pain, neck pain, dysmenorrhea, headache, toothache, sprains, strains, myositis, neuralgia, synovitis, gout, ankylosing spondylitis, bursitis, edema, inflammations following dental procedures, inflammations following dental procedures, vascular diseases, migraine headaches, periarteritis nodosa, thyroiditis, aplastic anemia, Hodkin's disease, sclerodoma, type I diabetes, myasthenia gravis, sarcoidosis, nephrotic syndrome, Behcet's syndrome, polymyositis, gingivitis, hypersensivity, conjunctivitis, swelling ocurring after injury and myocardia ischemia, for the prophylaxis and/or treatment of asthma, for the prophylaxis and/or treatment of bronchitis, for the prophylaxis and/or treatment of tendinitis, for the prophylaxis and/or treatment of bursitis, for the prophylaxis and/or treatment of skin related conditions, whereby said skin related conditions may preferably be selected from the group consisting of psoriasis, eczema, burns and dermatitis, for the prophylaxis and/or treatment of gastrointestinal disorders, whereby said gastrointestinal disorders may preferably be selected from the group consisting of inflammatory bowel disease, Crohn's disease, gastritis, irritable bowel syndrome and ulcerative colitis, or for treatment of fever, or for the prophylaxis and/or treatment of cancer or a cancer-related disorders, whereby said cancer or related disorder may preferably be selected from the group consisting of brain cancer, bone cancer, epithelial cell-derived neoplasia (epithelial carcinoma), basal cell carcinoma, adenocarcinoma, gastrointestinal cancer, lip cancer, colon cancer, liver cancer, bladder cancer, pancreas cancer, ovary cancer, cervical cancer, lung cancer, breast cancer, skin cancer, squamous cell cancer, prostate cancer, renal cell carcinoma and other known cancers that effect epithelial cells throughout the body, for the prophylaxis and/or treatment of polyps, for the prophylaxis and/or treatment of angiogenesis mediated disorders, preferably selected from the group consisting of metastasis, corneal graft rejection, ocular neovascularization, retinal neovascularisation, diabethic retinopathy, retrolental fibroplasia, neovascular glaucoma, gastric ulcer, infantile hemaginomas, angiofibroma of the nasopharynx, avascular necrosis of the bone and endometriosis.

More preferably the inventive medicament is suitable for the treatment of pain, whereby said pain is preferably selected from the group consisting of neuropathic pain, acute pain, chronic pain, post-operative pain, chronic lower back pain, cluster headaches, herpes neuralgia, phantom limb pain, central pain, dental pain, resistant pain, visceral pain, surgical pain, bone injury pain, pain during labor and delivery, pain resulting from burns, pain resulting from sunburns, post partum pains, migraine, angina pain, genitourinary tract-related pain, pain from cystitis and nociceptive pain, for the prophylaxis and/or treatment of inflammation and/or for the prophylaxis and/or treatment of inflammation related disorders, whereby said inflammation-related disorders may preferably be selected from the group consisting of arthritis, rheumatoid arthritis, spondyloarthropathies, gouty arthritis, osteoarthritis, systemic lupus erythematosus, juvenile arthritis, rheumatic fever, symptoms associated with influenza or other viral infections, common cold, lower back pain, neck pain, dysmenorrhea, headache, toothache, sprains, strains, myositis, neuralgia, synovitis, gout, ankylosing spondylitis, bursitis, edema, inflammations following dental procedures, inflammations following dental procedures, vascular diseases, migraine headaches, periarteritis nodosa, thyroiditis, aplastic anemia, Hodkin's disease, sclerodoma, type I diabetes, myasthenia gravis, sarcoidosis, nephrotic syndrome, Behcet's syndrome, polymyositis, gingivitis, hypersensivity, conjunctivitis, swelling ocurring after injury and myocardia ischemia and/or for the prophylaxis and/or treatment of urinary incontinence.

Those skilled in the art understand that the components (A) and (B) of the active substance combination according to the present invention may be administered simultaneously or sequentially to one another, whereby in each case components (A) and (B) may be administerd via the same or different administration pathways, e.g. orally or parenterally preferably both components (A) and (B) are administered simultaneously in one and the same administration form.

Another aspect of the present invention relates to the use of an inventive active substance combination for the manufacture of a medicament for the treatment of pain, whereby said pain is preferably selected from the group consisting of neuropathic pain, acute pain, chronic pain, post-operative pain, chronic lower back pain, cluster headaches, herpes neuralgia, phantom limb pain, central pain, dental pain, resistant pain, visceral pain, surgical pain, bone injury pain, pain during labor and delivery, pain resulting from burns, pain resulting from sunburns, post partum pains, migraine, angina pain, genitourinary tract-related pain, pain from cystitis and nociceptive pain, for the prophylaxis and/or treatment of urinary incontinence, for the prophylaxis and/or treatment of neurogenic inflammation for the prophylaxis and/or treatment of depression, for the prophylaxis and/or treatment of inflammation and/or for the prophylaxis and/or treatment of inflammation related disorders, whereby said inflammation-related disorders may preferably be selected from the group consisting of arthritis, rheumatoid arthritis, spondyloarthropathies, gouty arthritis, osteoarthritis, systemic lupus erythematosus, juvenile arthritis, rheumatic fever, symptoms associated with influenza or other viral infections, common cold, lower back pain, neck pain, dysmenorrhea, headache, toothache, sprains, strains, myositis, neuralgia, synovitis, gout, ankylosing spondylitis, bursitis, edema, inflammations following dental procedures, inflammations following dental procedures, vascular diseases, migraine headaches, periarteritis nodosa, thyroiditis, aplastic anemia, Hodkin's disease, sclerodoma, type I diabetes, myasthenia gravis, sarcoidosis, nephrotic syndrome, Behcet's syndrome, polymyositis, gingivitis, hypersensivity, conjunctivitis, swelling ocurring after injury and myocardia ischemia, for the prophylaxis and/or treatment of asthma, for the prophylaxis and/or treatment of bronchitis, for the prophylaxis and/or treatment of tendinitis, for the prophylaxis and/or treatment of bursitis, for the prophylaxis and/or treatment of skin related conditions, whereby said skin related conditions may preferably be selected from the group consisting of psoriasis, eczema, burns and dermatitis, for the prophylaxis and/or treatment of gastrointestinal disorders, whereby said gastrointestinal disorders may preferably be selected from the group consisting of inflammatory bowel disease, Crohn's disease, gastritis, irritable bowel syndrome and ulcerative colitis, or for treatment of fever, or for the prophylaxis and/or treatment of cancer or a cancer-related disorders, whereby said cancer or related disorder may preferably be selected from the group consisting of brain cancer, bone cancer, epithelial cell-derived neoplasia (epithelial carcinoma), basal cell carcinoma, adenocarcinoma, gastrointestinal cancer, lip cancer, colon cancer, liver cancer, bladder cancer, pancreas cancer, ovary cancer, cervical cancer, lung cancer, breast cancer, skin cancer, squamous cell cancer, prostate cancer, renal cell carcinoma and other known cancers that effect epithelial cells throughout the body, for the prophylaxis and/or treatment of polyps, for the prophylaxis and/or treatment of angiogenesis mediated disorders, preferably selected from the group consisting of metastasis, corneal graft rejection, ocular neovascularization, retinal neovascularisation, diabethic retinopathy, retrolental fibroplasia, neovascular glaucoma, gastric ulcer, infantile hemaginomas, angiofibroma of the nasopharynx, avascular necrosis of the bone and endometriosis.

The use of an inventive active substance combination for the preparation of a medicament for the treatment of pain, whereby said pain is preferably selected from the group consisting of neuropathic pain, acute pain, chronic pain, post-operative pain, chronic lower back pain, cluster headaches, herpes neuralgia, phantom limb pain, central pain, dental pain, resistant pain, visceral pain, surgical pain, bone injury pain, pain during labor and delivery, pain resulting from burns, pain resulting from sunburns, post partum pains, migraine, angina pain, genitourinary tract-related pain, pain from cystitis and nociceptive pain, for the prophylaxis and/or treatment of inflammation and/or for the prophylaxis and/or treatment of inflammation related disorders, whereby said inflammation-related disorders may preferably be selected from the group consisting of arthritis, rheumatoid arthritis, spondyloarthropathies, gouty arthritis, osteoarthritis, systemic lupus erythematosus, juvenile arthritis, rheumatic fever, symptoms associated with influenza or other viral infections, common cold, lower back pain, neck pain, dysmenorrhea, headache, toothache, sprains, strains, myositis, neuralgia, synovitis, gout, ankylosing spondylitis, bursitis, edema, inflammations following dental procedures, inflammations following dental procedures, vascular diseases, migraine headaches, periarteritis nodosa, thyroiditis, aplastic anemia, Hodkin's disease, sclerodoma, type I diabetes, myasthenia gravis, sarcoidosis, nephrotic syndrome, Behcet's syndrome, polymyositis, gingivitis, hypersensivity, conjunctivitis, swelling ocurring after injury and myocardia ischemia and/or for the prophylaxis and/or treatment of urinary incontinence is particularly preferred.

Yet another aspect of the present invention related to pharmaceutical formulations in different pharmaceutical forms comprising an inventive active substance combination and optionally at least one further active substance and/or optionally at least one auxiliary substance.

Preferably the inventive pharmaceutical formulation is suitable for oral or parenteral administration, more preferably for oral, intravenous, intraperitoneal, intramuscular, subcutaneous, intrathekal, rectal, transdermal, transmucosal or nasal administration. Inventive pharmaceutical formulation for oral administration are preferably selected from the group consisting of tablets, drageés, capsules, drops, gels, juices, sirups, solutions and suspensions.

The pharmaceutical formulation of the present invention for oral administration may also be in the form of multiparticulates, preferably pellets or granules, optionally compressed into a tablet, filled into a capsule or suspended in a suitable liquid. Suitable liquids are known to those skilled in the art.

The inventive pharmaceutical formulations may—depending on their route of administration—also contain one or more auxiliary substances known to those skilled in the art. The pharmaceutical formulations according to the present invention may be produced according to standard procedures known to those skilled in the art, e.g. from the tables of contents from “Pharmaceutics: the Science of Dosage Forms”, Second Edition, Aulton, M. E. (Ed.) Churchill Livingstone, Edinburgh (2002); “Encyclopedia of Pharmaceutical Technology”, Second Edition, Swarbrick, J. and Boylan J. C. (Eds.), Marcel Dekker, Inc. New York (2002); “Modern Pharmaceutics”, Fourth Edition, Banker G. S. and Rhodes C. T. (Eds.) Marcel Dekker, Inc. New York 2002 and “The Theory and Practice of Industrial Pharmacy”, Lachman L., Lieberman H. and Kanig J. (Eds.), Lea & Febiger, Philadelphia (1986). The respective descriptions are incorporated by reference and are part of the present disclosure.

In one embodiment of the present invention the pharmaceutical formulation comprises one or both of the components (A) and (B) at least partially in a sustained-release form.

By incorporating one or both of these components at least partially or completely in a sustained-release form it is possible to extend the duration of their effect, allowing for the beneficial effects of such a sustained release form, e.g. the maintenance of even concentrations in the blood.

Suitable sustained-release forms as well as materials and methods for their preparation are known to those skilled in the art, e.g. from the tables of contents from “Modified-Release Drug Delivery Technology”, Rathbone, M. J. Hadgraft, J. and Roberts, M. S. (Eds.), Marcel Dekker, Inc., New York (2002); “Handbook of Pharmaceutical Controlled Release Technology”, Wise, D. L. (Ed.), Marcel Dekker, Inc. New York, (2000); “Controlled Drug Delivery”, Vol. I, Basic Concepts, Bruck, S. D. (Ed.), CRC Press Inc., Boca Raton (1983) and from Takada, K. and Yoshikawa, H., “Oral Drug delivery”, Encyclopedia of Controlled Drug Delivery, Mathiowitz, E. (Ed.), John Wiley & Sons, Inc., New York (1999), Vol. 2, 728-742; Fix, J., “Oral drug delivery, small intestine and colon”, Encylopedia of Controlled Drug Delivery, Mathiowitz, E. (Ed.), John Wiley & Sons, Inc., New York (1999), Vol. 2, 698-728. The respective descriptions are incorporated by reference and are part of the disclosure. If the pharmaceutical formulation according to the present invention comprises at least one of the components (A) and (B) at least partially in a sustained-release form, said sustained release may preferably be achieved by the application of at least one coating or provision of a matrix comprising at least one sustained-release material.

The sustained-release material is preferably based on an optionally modified, water-insoluble, natural, semisynthetic or synthetic polymer, or a natural, semisynthetic or synthetic wax or fat or fatty alcohol or fatty acid, or on a mixture of at least two of these afore mentioned components.

The water-insoluble polymers used to produce a sustained-release material are preferably based on an acrylic resin, which is preferably selected from the group of poly(meth)acrylates, particularly preferably poly(C₁₋₄)alkyl (meth)acrylates, poly(C₁₋₄)dialkylamino(C₁₋₄)alkyl (meth)acrylates and/or copolymers or mixtures thereof, and very particularly preferably copolymers of ethyl acrylate and methyl methacrylate with a monomer molar ratio of 2:1 (Eudragit NE30D®), copolymers of ethyl acrylate, methyl methacrylate and trimethylammonium ethyl methacrylate-chloride with a monomer molar ratio of 1:2:0.1 (Eudragit RS®), copolymers of ethyl acrylate, methyl methacrylate and trimethylammonium ethyl methacrylate-chloride with a monomer molar ratio of 1:2:0.2 (Eudragit RL®), or a mixture of at least two of the above-mentioned copolymers. These coating materials are commercially available as 30 wt. % aqueous latex dispersions, i.e. as Eudragit RS30D®, Eudragit NE30D® or Eudragit RL30D®, and may also be used as such for coating purposes.

In another embodiment, the sustained-release material is based on water-insoluble cellulose derivatives, preferably alkyl celluloses, particularly preferably ethyl cellulose, or cellulose esters, e.g. cellulose acetate. Aqueous ethyl cellulose dispersions are commercially available, for example, under the trademarks Aquacoat® or Surelease®.

As natural, semisynthetic or synthetic waxes, fats or fatty alcohols, the sustained-release material may be based on carnauba wax, beeswax, glycerol monostearate, glycerol monobehenate, glycerol ditripalmitostearate, microcrystalline wax, cetyl alcohol, cetylstearyl alcohol or a mixture of at least two of these components. The afore mentioned polymers of the sustained-release material may also comprise a conventional, physiologically acceptable plasticizer in amounts known to those skilled in the art.

Examples of suitable plasticizers are lipophilic diesters of a C₆-C₄₀ aliphatic or aromatic dicarboxylic acid and a C₁-C₈ aliphatic alcohol, e.g. dibutyl phthalate, diethyl phthalate, dibutyl sebacate or diethyl sebacate, hydrophilic or lipophilic citric acid esters, e.g. triethyl citrate, tributyl citrate, acetyltributyl citrate or acetyltriethyl citrate, polyethylene glycols, propylene glycol, glycerol esters, e.g. triacetin, Myvacet® (acetylated mono- and diglycerides, C₂₃H₄₄O₅ to C₂₅H₄₇O₇), medium-chain triglycerides (Miglyol®), oleic acid or mixtures of at least two of said plasticizers. Aqueous dispersions of Eudragit RS® and optionally Eudragit RL® preferably contain triethyl citrate. The sustained-release material may comprise one or more plasticisers in amounts of, for example, 5 to 50 wt. % based on the amount of polymer(s) used.

The sustained-release material may also contain other conventional auxiliary substances known to those skilled in the art, e.g. lubricants, coloured pigments or surfactants.

The pharmaceutical formulation of the present invention may also comprise at least one of the components (A) and (B) covered by an enteric coating form which dissolves as a function of pH. Because of this coating, part or all of the pharmaceutical formulation can pass through the stomach undissolved and the components (A) and/or (B) are only released in the intestinal tract. The enteric coating preferably dissolves at a pH of between 5 and 7.5.

The enteric coating may be based on any enteric material known to those skilled in the art, e.g. on methacrylic acid/methyl methacrylate copolymers with a monomer molar ratio of 1:1 (Eudragit L®), methacrylic acid/methyl methacrylate copolymers with a monomer molar ratio of 1:2 (Eudragit S®), methacrylic acid/ethyl acrylate copolymers with a monomer molar ratio of 1:1 (Eudragit L30D-55®), methacrylic acid/methyl acrylate/methyl methacrylate copolymers with a monomer molar ratio of 7:3:1 (Eudragit FS®), shellac, hydroxypropyl methyl cellulose acetate-succinates, cellulose acetate-phthalates or a mixture of at least two of these components, which can optionally also be used in combination with the above-mentioned water-insoluble poly(meth)acrylates, preferably in combination with Eudragit NE30D® and/or Eudragit RL® and/or Eudragit RS®.

The coatings of the pharmaceutical formulations of the present invention may be applied by the conventional processes known to those skilled in the art, e.g. from Johnson, J. L., “Pharmaceutical tablet coating”, Coatings Technology Handbook (Second Edition), Satas, D. and Tracton, A. A. (Eds), Marcel Dekker, Inc. New York, (2001), 863-866; Carstensen, T., “Coating Tablets in Advanced Pharmaceutical Solids”, Swarbrick, J. (Ed.), Marcel Dekker, Inc. New York (2001), 455-468; Leopold, C. S., “Coated dosage forms for colon-specific drug delivery”, Pharmaceutical Science & Technology Today, 2(5), 197-204 (1999), Rhodes, C. T. and Porter, S. C., Coatings, in Encyclopedia of Controlled Drug Delivery. Mathiowitz, E. (Ed.), John Wiley & Sons, Inc., New York (1999), Vol.1, 299-311. The respective descriptions are incorporated by reference and are part of the disclosure.

In another embodiment, the pharmaceutical formulation of the present invention contains one or both of components (A) and (B) not only in sustained-release form, but also in non-retarded form. By combination with the immediately released form, a high initial dose can be achieved for the rapid onset of the beneficial effect. The slow release from the sustained release form then prevents the beneficial effect from diminishing. Such a pharmaceutical formulation is particularly useful for the treatment of acute health problems.

This may be achieved, for example, by a pharmaceutical formulation having at least one immediate-release coating comprising at least one of the components (A) and (B) to provide for rapid onset of the beneficial effect after administration to the patient.

For example, an inventive pharmaceutical formulation suitable for the treatment of pain, may preferably comprise component (B) in immediate-relase form in addition to components (A) and (B) in sustained release form.

An inventive pharmaceutical formulation suitable for the treatment of inflammation and inflammation-related disorders may preferably comprise both components (A) and (B) each in immediate release form and in sustained release form.

It has surprisingly been found that the pharmacological efficacy, particularly the analgesic efficacy, of the inventive substance combination is maintained or even improved with respect to the administration of the NSAID component alone for comparable amounts to be administered, whereas the undesired side effects typically associated with the NSAIDs component, particularly with Cyclooxygenase-1 inhibitors and Cyclooxygenase-2 inhibitors of the first generartion are significantly reduced. Thus, the inventive active substance combination allows to make use of the many benefical effects associated with NSAIDs but without or at least to a significantly reduced extent having to cope with the disadvantages typically associated with their use.

Pharmacological Methods:

I. Determination of Analgesic Activity

Ia. Writhing Test in Mice

The Writhing test for the determination of the analgesic activity of the inventive active substance combination is carried out according to the method described in the publication of E. Siegmund et al., Proc. Soc. Exp. Biol. Med. 1957, 95, 729-731 using male Swiss albino mice (20-25 g body weight, obtained from Harlan, S. Feliu de Codinas, Spain). The respective part of the description is hereby incorporated by reference and forms part of the respective disclosure.

The Writhing reactions are induced by intraperitoneal injection of phenylbenzoquinone (25 ml/kg in a 0.02% (volume/volume) ethanolic solution in a 5% (volume/volume) solution in destillated water with Evan's blue in an amount of 0.1% weight/volume) and the writhing reactions are counted during a 15 minute period following the injection. The substances to be tested are orally administered 60 minutes prior to the injection of the phenylbenzoquinone solution. The percentage of the inhibition of the writhing reactions is calculated on the basis of the control group as basis for 0% inhibition.

Ib. Formaline Test in Mice

The Formaline test for the determination of the analgesic activity of the inventive active substance combination is carried out according to the method described in the publication of T. Ohkubo et al., J. Pharmacol. Exp. Ther. 1990, 252, 1261-1268 using male Swiss albino mice (20-25 g body weight, obtained from Harlan, S. Feliu de Codinas, Spain). The respective part of the description is hereby incorporated by reference and forms part of the respective disclosure.

The substances to be tested are intraperitoneally administered to the mice in 5% by weight solution of arabic gum in destined water as vehicle. 15 minutes later 20 μl of a 5% by weight solution of formaline in saline solution is injected into the back of the right paw of the animals. The total time in seconds of licking and/or biting the injected paw is registered in the acute phase, i.e. 0-5 minutes (phase I), and in the chronic phase, i.e. 15-30 (phase II), minutes after injection of the formaline. The percentage of inhibition is calculated based on the medium values of the acute and chronic phases of the control group as 0% inhibition of the primary and secondary response.

II. Determination of Ulcerogenic Effect in Rats

The ulcerogenic effect of the inventive active substance combination is determined in male Wistar albino rats (body weight 160-200 g, obtained from Harlan, S. Feliu de Codinas, Spain) according to the method described in the publication of J. L. Wallace et al., Am. J. Psychiol. 1990, 259, G462-G467. The respective part of the description is incorporated by reference and forms part of the present disclosure.

Prior to the tests the rats are kept in cages for 24 hours with free access to drinking water. Afterwards the substances to be tested are orally administered to the rats in form of a 5% by weight suspension in arabic gum. Three hours after the administration of the respective substances to be tested the rats are sacrificed by inhalation of carbon dioxide, the stomachs are removed, opened along the great curvature, washed with saline solution and extended over a suitable frame. By the use of a Projectt 1.2 image analyzer (Projectt, Barcelona, Spain) the ulcerated areas of the stomachs are determined and their size expressed in mm².

The present invention is illustrated below with the aid of examples. These illustrations are given solely by way of example and do not limit the general spirit of the present invention.

### EXAMPLES

General method for the preparation of an active substance combination salt:

The following salts were prepared according to the afore mentioned method:
(a) R-(+)-5-[α-[2-(Dimethylamino)ethoxy]benzyl]-1-methyl-1H-pyrazole naproxenate (hereinafter referred to as R-(+)-Cizolirtine naproxenate)
(b) S-(−)-5-[α-[2-( Dimethylamino)ethoxy]benzyl]-1-methyl-1H-pyrazole naproxenate (hereinafter referred to as R-(+)-Cizolirtine naproxenate)
(c) R-(+)-5-[α-[2-(Dimethylamino)ethoxy]benzyl]-1-methyl-1H-pyrazole diclofenacate (hereinafter referred to as R-(+)-Cizolirtine diclofenacate)
(d) S-(−)-5-[α-[2-(Dimethylamino)ethoxy]benzyl]-1-methyl-1H-pyrazole diclofenacate (hereinafter referred to as R-(+)-Cizolirtine diclofenacate)
(e) R-(+)-5-[α-[2-(Dimethylamino)ethoxy]benzyl]-1-methyl-1H-pyrazole S-(+)-ibuprofenate (hereinafter referred to as R-(+)-Cizolirtine ibuprofenate)
(f) S-(−)-5-[α-[2-(Dimethylamino)ethoxy]benzyl]-1-methyl-1H-pyrazole S-(+) ibuprofenate (hereinafter referred to as R-(+)-Cizolirtine ibuprofenate)

The molecular weights of Cizolirtine in form of its free base (259 g/mol), Naproxen (252 g/mol), Diclofenac (273 g/mol) and Ibuprofen (206 g/mol) are comparable. Thus, the pharmacological tests according to the present examples have been carried out using identical dosages, such as 40 mg/kg, 80 mg/kg or 160 mg/kg.

### Example 1

The active substance combination salts (a) and (b) as well as their respective components, i.e. naproxen, R-(+)5-[α-[2-(Dimethylamino)ethoxy]benzyl]-1-methyl-1H-pyrazole (hereinafter referred to as R-(+)-cizolirtine) and S-(−)-5-[α-[2-(Dimethylamino)ethoxy]benzyl]-1-methyl-1H-pyrazole (hereinafter referred to as S-(−)-cizolirtine) were tested for their analgesic activity and their ulcerogenic effects. The respective results are given in the following tables A, B and C.

TABLE A
Writhing test

| Substance | % Activity | | | | |
|---|---|---|---|---|---|
| tested | Dosis (p.o.) | 40 mg/kg | 80 mg/kg | 160 mg/kg | ED₅₀ |
| Naproxen | | 32.0 | 51.2 | 78.5 | 71.22 |
| R-(+)- | | 29.0 | 49.7 | 72.5 | 78.99 |
| Cizolirtine | | | | | |
| S-(−)- | | 29.0 | 49.7 | 59.7 | 95.26 |
| Cizolirtine | | | | | |
| R-(+)- | | 32.0 | 52.0 | 77.3 | 71.29 |
| Cizolirtine | | | | | |
| naproxenate | | | | | |
| S-(−)- | | 26.7 | 52.4 | 68.5 | 82.15 |
| Cizolirtine | | | | | |
| naproxenate | | | | | |

TABLE B
Formaline test

| | |
|---|---|
| Dosis | % Activity |

| Substance tested | (mg/kg, i.p.) | Phase I | Phase II |
|---|---|---|---|
| Naproxen | 40 | 31.5 | 18 |
| R-(+)-Cizolirtine | 40 | 83 | 68.5 |
| R-(+)-Cizolirtine | 40 | 49 | 36 |
| naproxenate | | | |

TABLE C
Ulcerogenic effects

| | Ulcerated area (mm²) | | |
|---|---|---|---|
| Substance tested | dosis (mg/kg, p.o.) | 80 | 160 |
| Naproxen | | 10.5 | |
| R-(+)-Cizolirtine | | | 6.86 |
| naproxenate | | | |

### Example 2

The active substance combination salt (c) as well as its respective components, i.e. diclofenac in form of its sodium salt and R-(+)-5-[α-[2-(Dimethylamino)ethoxy]benzyl]-1-methyl-1H-pyrazole (hereinafter referred to as R-(+)-cizolirtine) were tested for their analgesic activity and their ulcerogenic effects. The respective results are given in the following tables D and E.

TABLE D
Writhing test

| | % Activity | | | | |
|---|---|---|---|---|---|
| Substance | Dosis | | | | |
| tested | (p.o.) | 40 mg/kg | 80 mg/kg | 160 mg/kg | ED₅₀ |
| Diclofenac- | | 19.8 | 54.9 | 84.1 | 75.1 |
| sodium | | | | | |
| R-(+)- | | 28.9 | 49.7 | 72.5 | 79 |
| Cizolirtine | | | | | |
| R-(+)- | | 14.7 | 57.3 | 72.1 | 84 |
| Cizolirtine | | | | | |
| diclofenacate | | | | | |

TABLE E
Ulcerogenic effects

| | Ulcerated area (mm²) | | | |
|---|---|---|---|---|
| Susbstance tested | dosis (mg/kg, p.o.) | 40 | 80 | 160 |
| diclofenac-sodium | | 3.81 | 13.7 | |
| R-(+)-Cizolirtine | | | 2.75 | 7.24 |
| diclofenacate | | | | |

### Example 3

The active substance combination salt (e) as well as its respective components, i.e. ibuprofen or ibuprofen-sodium and R-(+)-5-[α-[2-(Dimethylamino)ethoxy]benzyl]-1-methyl-1H-pyrazole (hereinafter referred to as R-(+)-cizolirtine) were tested for their analgesic activity and their ulcerogenic effects. The respective results are given in the following tables F and G.

TABLE F
Writhing test

| | % Activity | | | | |
|---|---|---|---|---|---|
| Substance | Dosis | | | | |
| tested | (p.o.) | 40 mg/kg | 80 mg/kg | 160 mg/kg | ED₅₀ |
| Ibuprofen | | 19.8 | 47.6 | 62.9 | 102.6 |
| R-(+)- | | 28.9 | 49.7 | 72.5 | 79 |
| Cizolirtine | | | | | |
| R-(+)- | | 15.9 | 45 | 80.9 | 84.8 |
| Cizolirtine | | | | | |
| ibuprofenate | | | | | |

TABLE G
Ulcerogenic effects

| | Ulcerated area (mm²) | | |
|---|---|---|---|
| Substance tested | dosis (mg/kg, p.o.) | 80 | 160 |
| Ibuprofen-sodium | | 5.29 | |
| R-(+)-Cizolirtine | | | 3.21 |
| ibuprofenate | | | |

As can be seen from the examples 1-3 the inventive active substance combination salts show similar or even improved analgesic activity compared to the respective non-steroidal anti-inflammatory drug component alone, whereas the ulcerogenic effect usually associated with the administration of such an NSAID component is significantly reduced.

In this context it is important to note that in order to compare the ulcerogenic effect of a certain amount of an NSAID component it has to be compared with the value obtained for twice this amount of an active substance combination salt, since only half the amount of the salt is made up from the respective NSAID component.

1. Active substance combination comprising
(A) at least one substituted carbinol compound of general formula I, 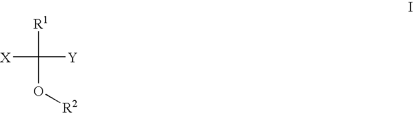
wherein
R¹ represents a hydrogen atom, a linear or branched alkyl radical, a linear or branched alkenyl radical, an optionally at least mono-substituted cycloaliphatic radical, which may contain at least one nitrogen atom as ring member, or a phenyl radical,
R² represents a hydrogen atom, an optionally at least one nitrogen atom as ring member containing cycloaliphatic radical, which may be at least mono-substituted by a linear or branched alkyl radical and/or which may be bound via a linear or branched alkylene group, an NR³R⁴-moiety, which is bound via a linear or branched alkylene group, or an NR⁵R⁶-moiety, which is bound via a linear or branched alkylene group,
R³ and R⁴, identical or different, represent a linear or branched alkyl radical or an unsubstituted benzyl radical,
R⁵ and R⁶ together with the bridging nitrogen atom represent a saturated, unsubstituted, optionally at least one further heteroatom as ring member containing heterocyclic radical,
X represents an optionally at least mono-substituted phenyl radical or an optionally at least mono-substituted thienyl radical, wherein in each case the substituents may be independently selected from the group consisting of a linear or branched alkyl radical, a linear or branched alkoxy group, a linear or branched alkyl radical, which is at least partially halogenated and a halogen atom,
Y represents a heteroaryl radical, which contains one or more nitrogen atoms as ring members and which is unsubstituted or at least mono-substituted by one or more substitutents independently from one another selected from the group consisting of a halogen atom, a linear or branched alkyl radical, a benzyl radical, a ciano group bound via a linear or branched C₁₋₄-alkylene group, a carboxy group bound via a linear or branched C₁₋₄-alkylene group, a methoxy carbonyl group bound via a linear or branched C:L₄-alkylene group, a hydroxy group bound via a linear or branched C₁₋₄-alkylene group, an amino group bound via a linear or branched C₁₋₄-alkylene group, a (C₁₋₄) dialkylamino group bound via a linear or branched C₁₋₄-alkylene group, and a cycloaliphatic radical, which contains at least one nitrogen atom as ring member and which is bound via a linear or branched C₁₋₄-alkylene group, or Y represents an unsubstituted heteroaryl radical, which contains two nitrogen atoms as ring members and which is condensed with a saturated, one methyl-substituted nitrogen atom as ring member containing cycloaliphatic group,
optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate,
and
(B) optionally at least one non-steroidal anti-inflammatory drug (NSAID).

2. Active substance combination according to claim 1, characterized in that R¹ represents a hydrogen atom, a linear or branched C₁₋₄ alkyl radical, a linear or branched C₂₋₄ alkenyl radical, a 5- or 6-membered cycloaliphatic radical, which may contain at least one nitrogen atom as ring member and/or which may be at least mono-substituted by a linear or branched C₁₋₄ alkyl radical, or a phenyl radical, preferably a hydrogen atom, a linear or branched C₁₋₄ alkyl radical, a vinyl group, a cyclohexyl radical, an N-Methyl-piperidyl radical or a phenyl radical.

3. Active substance combination according to claim 1, characterized in that R² represents a hydrogen atom, an optionally at least one nitrogen atom as ring member containing, 5- or 6-membered cycloaliphatic radical, which may be at least mono-substituted by a linear or branched C₁₋₄-alkyl radical and/or which may be bound via a linear or branched C₁₋₄-alkylene group, a NR³R⁴-moiety, which is bound via a linear or branched C₁₋₄ alkylene group, or a NR⁵R⁶-moiety, which is bound via a linear or branched C₁₋₄ alkylene group, preferably a hydrogen atom, an optionally at least one nitrogen atom as ring member containing, 5- or 6-membered cycloaliphatic radical, which may be at least mono-substituted by a linear or branched C₁₋₄-alkyl radical and/or which may be bound via a linear or branched C₁₋₄-alkylene group, a NR³R⁴-moiety, which is bound via a linear or branched C₂₋₃ alkylene group, or a NR⁵R⁶-moiety, which is bound via a linear or branched C₂₋₃ alkylene group.

4. Active substance combination according to claim 1, characterized in that R³ and R⁴, identical or different, independently from one another represent a linear or branched C₁₋₄ alkyl radical or an unsubstituted benzyl radical, preferably a linear or branched C₁₋₄ alkyl radical.

5. Active substance combination according to claim 1, characterized in that R⁵ and R⁶ together with the bridging nitrogen atom represent a saturated, unsubstituted, optionally at least one oxygen atom as ring member containing, 5- or 6-membered heterocyclic radical.

6. Active substance combination according to claim 1, characterized in that X represents an optionally at least mono-substituted phenyl radical or an optionally at least mono-substituted thienyl radical, wherein in each case the substituents may be independently selected from the group consisting of a linear or branched C₁₋₄ alkyl radical, a linear or branched C₁₋₄ alkoxy radical, a linear or branched C₁₋₄ alkyl radical, which is at least partially fluorinated, a fluorine atom, a chlorine atom and a bromine atom, preferably represents an optionally at least mono-substituted phenyl radical or an optionally at least mono-substituted thienyl radical, wherein in each case the substituents may be independently selected from the group consisting of a methyl radical, a methoxy radical, a trifluoromethyl radical, a fluorine atom, a chlorine atom and a bromine atom.

7. Active substance combination according to claim 1, characterized in that Y represents an azole radical selected from the group consisting of
a) a pyrazole of the general formula (a): 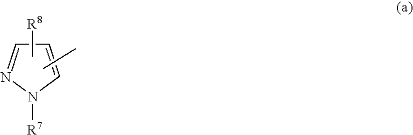
in which R⁷ represents a linear or branched C₁₋₁₂ alkyl radical, a benzyl radical or a radical of the type: 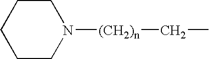
in which n=1 or 2, and
R⁸ represents a hydrogen atom, a methyl radical or a halogen atom, preferably a hydrogen atom, a methyl radical, a bromine atom or a chlorine atom,
b) an imidazole of the general formula 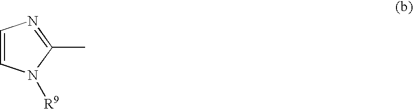
in which R⁹ represents a hydrogen atom, a C₁₋₁₂ alkyl radical, a benzyl radical, or a radical of the general formula (b1):
R¹⁰—(CH₂)ₙ— (b1)
in which n is 2, 3 or 4 and R¹⁰ represents a piperidinyl radical, a phenyl radical, a cyano group, a hydroxyl radical, a carboxy radical, an amino group, a dimethylamino group, or a methyl ester (CH₃—O—C(═O)—) group,
and
(c) an imidazole of the following formula:

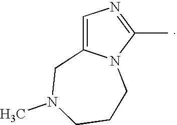

8. Active substance combination according to claim 1, characterized in that as component (A) at least one carbinol compound of general formula I 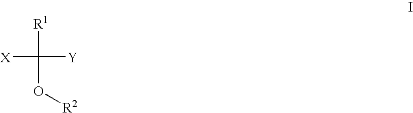
is present, wherein
R¹ represents a hydrogen atom, a methyl radical, an ethyl radical, an n-propyl radical, an iso-propyl radical, a sec-butyl radical, a tert-butyl radical, an n-butyl radical, a vinyl radical, a cyclohexyl radical, an N-methyl-piperidinyl group, or a phenyl group,
R² represents a hydrogen atom, a dimethylaminoethyl group, a pyrrolidinylethyl group, a piperidinylethyl group, a methyl-benzyl-aminoethyl group, a morpholinylethyl group, a diisopropylaminoethyl group, a dimethylaminopropyl group, a piperidinylpropyl group, a pyrrolidinylpropyl group, a morpholinylpropyl group, an N-methyl-2-piperidyl group, an N-ethyl-2-piperidyl group, an N-propyl-2-piperidyl group, an N-methyl-2-pyrrolidinyl group, an N-ethyl-2-pyrrolidinyl group, an N-propyl-2-pyrrolidinyl group, or a 2-dimethylaminoethyl-1-methyl group,
X represents a phenyl radical, a 2-methyl-phenyl radical, a 3-methyl-phenyl radical, a 4-methyl phenyl radical, a 2-chloro-phenyl radical, a 3-chloro-phenyl radical, a 4-chloro-phenyl radical, a 2-fluoro-phenyl radical, a 3-fluoro-phenyl radical, a 4-fluoro-phenyl radical, a 2-trifluoromethyl-phenyl radical, a 3-trifluoromethyl-phenyl radical, a 4-trifluoromethyl-phenyl radical, a 2-methoxy-phenyl radical, a 3-methoxy-phenyl radical, a 4-methoxy-phenyl radical, a 3,4,5-tris-methoxy phenyl radical, a 3,4-dichloro-phenyl radical, a 2,4-dichloro-phenyl radical, a thien-2-yl radical, a thien-3-yl radical, a 3-methyl-thien-2-yl radical, a 5-methyl-thien-2-yl-radical, a 5-bromo-thien-2-yl radical or a 4-bromo-thien-2-yl-radical,
Y represents an azole radical selected from the group consisting of
a) a pyrazole of the general formula (a): 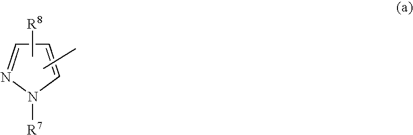
in which
R⁷ represents a methyl radical, an ethyl radical, an n-propyl radical, an iso-propyl radical, an n-butyl radical, a sec-butyl radical or a tert-butyl radical,
R⁸ represents a hydrogen atom, a methyl radical, a bromine atom or a chlorine atom,
b) an imidazole of the general formula 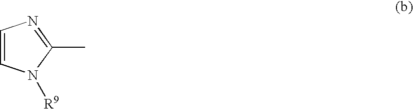
in which R⁹ represents a hydrogen atom, a methyl radical, an ethyl radical, an n-propyl radical, an iso-butyl radical, an n-butyl radical, a sec-butyl radical a tert-butyl radical, an n-pentyl radical, an n-hexyl radical, an n-heptyl radical, an n-octyl radical, an n-nonyl radical, an n-decyl radical, an n-undecyl radical an n-dodecyl radical, a benzyl radical, or a radical of the general formula (b1):
R¹⁰—(CH₂)ₙ— (b1)
in which n is 2, 3 or 4 and R¹⁰ represents a piperidinyl radical, a phenyl radical, a cyano group, a hydroxyl radical, a carboxy radical, an amino group, a dimethylamino group, or a methyl ester group,
and
(c) an imidazole of the following formula: 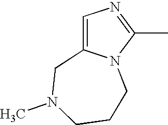
optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate.

9. Active substance combination according to claim 1, characterised in that as component (A) one or more compounds selected from the group consisting of
[1] 2-{α-[2-(dimethylamino)ethoxy]benzyl}-1-methyl-1H-imidazole,
[2] 2-{4-chloro-α-[2-(dimethylamino)ethoxy]-α-methylbenzyl}-1-methyl-1H-imidazole,
[3] 2-{4-chloro-α-[2-(dimethylamino)ethoxy]benzyl}-1-methyl-1H-imidazole,
[4] 2-{3-chloro-α-[2-(dimethylamino)ethoxy]benzyl}-1-methyl-1H-imidazole,
[5] 2-{4-chloro-α-[2-(dimethylamino)ethoxy]-α-methylbenzyl}-1-methyl-1H-imidazole,
[6] 2-{4-fluoro-α-[2-(dimethylamino)ethoxy]-α-methylbenzyl}-1-methyl-1H-imidazole,
[7] 2-{α-[2-(dimethylamino)ethoxy]-α-methyl-3-(trifluoromethyl)benzyl}-1-methyl-1H-imidazole,
[8] 2-{3-chloro-α-[2-(dimethylamino)ethoxy]-α-methylbenzyl}-1-methyl-1H-imidazole,
[9] 2-{3-chloro-α-[2-(dimethylamino)ethoxy]-α-propylbenzyl}-1-methyl-1H-imidazole,
[10] 1-butyl-2-{4-chloro-α-[2-(dimethylamino)ethoxy]-α-methylbenzyl}-1H-imidazole,
[11] 2-{α-[2-(dimethylamino)ethoxy]-α-methyl-4-methoxybenzyl)-1-methyl-1H-imidazole,
[12] 2-{3-chloro-α-methyl-α-[2-(N-pyrrolidinyl)ethoxy]benzyl}-1-methyl-1H-imidazole,
8 13] 2-{α-[2-(dimethylamino)ethoxyl-α-propyl-3,4,5-trimethoxybenzyl}-1-dodecyl-1H-imidazole,
[14] 1-butyl-2-{α-[2-(dimethylamino)ethoxy]-4-(trifluoromethyl)benzyl}-1H-imidazole,
[15] 1-methyl-2-{α-methyl-α-[2-(N-piperidyl)ethoxy]-3-(trifluoromethyl)benzyl}-1H-imidazole,
[16] 2-{α-cyclohexyl-3,4-dichloro-α-[2-(dimethylamino)ethoxy]benzyl}-1-methyl-1H-imidazole,
[17] 2-{3,4-dichloro-α-[2-(dimethylamino)ethoxy]-α-propylbenzyl}-1-methyl-1H-imidazole,
[18] 2-{3,4-dichloro-α-[2-(dimethylamino)ethoxy]-α-methylbenzyl}-1-methyl-1H-imidazole,
[19] 2-{3,4-dichloro-α-[2-(dimethylamino)ethoxy]benzyl}-1-methyl-1H-imidazole,
[20] 2-{4-chloro-α-[2-(dimethylamino)ethoxy]-α-methylbenzyl}-1-[2-(N-piperidyl)ethyl]-1H-imidazole,
[21] 2-{4-chloro-α-[2-(dimethylamino)ethoxy]-α-methylbenzyl}-1-[2-(N-piperidyl)propyl]-1H-imidazole,
[22] 1-(3-cyanopropyl)-2-{4-chloro-α-[2-(dimethylamino)ethoxy]benzyl}-1H-imidazole,
[23] 2-{4-chloro-α-[2-(dimethylamino)ethoxy]-α-(N-methyl-4-piperidyl)benzyl}-1-methyl-1H-imidazole,
[24] 1-benzyl-2-{α-[2-(N-benzyl-N-methylamino)ethoxy)-4-chlorobenzyl}-1H-imidazole,
[25] 2-{4-chloro-α-[2-(dimethylamino)ethoxy]-α-methylbenzyl}-7-methyl-6,7,8,9-tetrahydro-1H-imidazole[1,5-a][1,4]diazepine,
[26] 2-{4-chloro-α-[2-(dimethylamino)ethoxy]benzyl}-7-methyl-6,7,8,9-tetrahydro-1H-imidazole[1,5-a] [1,4]diazepine,
[27] 1-butyl-5-{α-[2-(dimethylamino)ethoxy]benzyl}-1H-pyrazole,
[28] 5-{α-(4-chlorophenyl) -α-[2-(dimethylamino)ethoxy]benzyl}-1-methyl-1H-pyrazole,
[29] 1-butyl-5-{α-[2-(dimethylamino)ethoxy]-3,4,5-trimethoxybenzyl}-1H-pyrazole,
[30] 1-butyl-5-{4-chloro-α-[2-(dimethylamino)ethoxy]-α-methylbenzyl}-1H-pyrazole,
[31] 5-{α-[2-(dimethylamino)ethoxy]benzyl}-1-methyl-1H-pyrazole,
[32] 5-{α-[2-(dimethylamino)ethoxy]-α-methylbenzyl}-1-methyl-1H-pyrazole,
[33] 5-{α- [2-(dimethylamino)ethoxy]-3,4,5-trimethoxybenzyl}-1-methyl-1H-pyrazole,
[34] 1-methyl-5-{α-[2-(N-pyrrolidinyl)ethoxy]benzyl)-1H-pyrazole,
[35] 1-methyl-5-{α-[2-(N-morpholinyl)ethoxy]benzyl}-1H-pyrazole,
[36] 5-{α-[2-(dimethylamino)ethoxy]-α-methyl-3,4,5-trimethoxybenzyl}-1-methyl-1H-pyrazole,
[37] 4-bromo-5-{α-[2-(dimethylamino)ethoxy]benzyl}-1-methyl-1H-pyrazole,
[38] 1,3-dimethyl-5-{α-[2-(dimethylamino)ethoxy)-α-methylbenzyl}-1H-pyrazole,
[39] 1,3-dimethyl-5-{a-[2-(dimethylamino)ethoxy]benzyl}-1H-pyrazole,
[40] 5-{a-[2-(dimethylamino)ethoxy]-2-methylbenzyl}-1-methyl-1H-pyrazole,
[41] 4-chloro-5-{4-chloro-α-[2-(dimethylamino)ethoxy)benzyl}-1-methyl-1H-pyrazole,
[42] 5-{4-chloro-α-[2-(dimethylamino)ethoxy]benzyl}-1-methyl-1H-pyrazole,
[43] 5-{3-chloro-α-[2-(dimethylamino)ethoxy]benzyl}-1-methyl-1H-pyrazole,
[44] 5-{α-[2-(dimethylamino)ethoxy]-4-methylbenzyl}-1-methyl-1H-pyrazole,
[45] 5-{2-chloro-α-[2-(dimethylamino)ethoxy]benzyl}-1-methyl-1H-pyrazole,
[46] 1-methyl-5-{α-[2-(N-piperidyl)ethoxy]benzyl}-1H-pyrazole,
[47] 1-methyl-5-{α-[2-(N-propyl-2-piperidyl)ethoxy]benzyl}-1H-pyrazole,
[48] 5-{α-[2-(N-ethyl-2-piperidyl)ethoxylbenzyl}-1-methyl-1H-pyrazole,
[49] 1-methyl-5-{α-[2-(N-methyl-2-pyrrolidinyl)ethoxy]benzyl}-1H-pyrazole,
[50] 5-{α-[2-(diisopropylamino)ethoxy]benzyl}-1-methyl-1H-pyrazole,
[51] 1-methyl-5-{α-[2-(N-methyl-2-piperidyl)ethoxyl]benzyl}-1H-pyrazole,
[52] 2-{4-chloro-α-[3-(dimethylamino)propoxy]-α-methylbenzyl}-1-methyl-1H-imidazole,
[53] 2-{3-chloro-α-[3-(dimethylamino)propoxy]benzyl}-1-methyl-1H-imidazole,
[54] 2-{4-chloro-α-[3-(dimethylamino)propoxy)-α-ethylbenzyl}-1-methyl-1H-imidazole,
[55] 2-{α-butyl-3-chloro-α-[3-(dimethylamino)propoxy]benzyl}-1-methyl-1H-imidazole,
[56] 2-{α-cyclohexyl-4-chloro-α-[3-(dimethylamino)propoxy]benzyl}-1-methyl-1H-imidazole,
[57] 2-{α-[3-(dimethylamino)propoxy]-4-fluoro-α-methylbenzyl}-1-methyl-1H-imidazole,
[58] 2-{α-[3-(dimethylamino)propoxy) -α-methyl-3-(trifluoromethyl)benzyl}-1-methyl-1H-imidazole,
[59] 2-{2-chloro-α-[3-(dimethylamino)propoxy]-α-methylbenzyl}-1-methyl-1H-imidazole,
[60] 2-{3-chloro-α-[3-(dimethylamino)propoxyl-α-methylbenzyl}-1-methyl-1H-imidazole,
[61] 2-{α-[3-(dimethylamino)propoxy]-α-methyl-3,4,5-trimethoxybenzyl}-1-methyl-1H-imidazole,
[62] 2-{α-[3-(dimethylamino)propoxy]-α-methyl-4-methoxybenzyl}-1-methyl-1H-imidazole,
[63] 2-{4-chloro-α-[3-(dimethylamino)propoxy]benzyl}-1-methyl-1H-imidazole,
[64] 2-{α-[3-(dimethylamino)propoxy]-3,4,5-trimethoxybenzyl}-1-methyl-1H-imidazole,
[65] 2-{α-[3-(dimethylamino)propoxy]-α-methyl-4-(trifluoromethyl)benzyl}-1-methyl-1H-imidazole,
[66] 2-{α-[3-(dimethylamino)propoxy)-3-(trifluoromethyl)benzyl)-1-methyl-1H-imidazole,
[67] 2-{α-[3-(dimethylamino)propoxy)-4-(trifluoromethyl)benzyl}-1-methyl-1H-imidazole,
[68] 2—{α-[3-(dimethylamino)propoxy]-4-methoxybenzyl}-1-methyl-1H-imidazole,
[69] 2-{α-butyl-α-[3-(dimethylamino)propoxy]-3-(trifluoromethyl)benzyl}-1-methyl-1H-imidazole,
[70] 1-butyl-2-{4-chloro-α-[3-(dimethylamino)propoxy)-α-methylbenzyl}-1H-imidazole,
[71] 1-butyl-2-{α-butyl-α-[3-(dimethylamino)propoxy]-3,4,5-trimethoxybenzyl}-1H-imidazole,
[72] 1-butyl-2-{α-butyl-2-chloro-α-[3-(dimethylamino)propoxy]benzyl}-1H-imidazole,
[73] 1-butyl-2-{α-butyl-2,4-dichloro-α-[3-(dimethylamino)propoxy]benzyl}-1H-imidazole,
[74] 1-butyl-2-{α-[3-(dimethylamino)propoxy]-4-(trifluoromethyl)benzyl}-1H-imidazole,
[75] 2-{4-chloro-α-[3-(N-piperidyl)propoxy]benzyl}-1-methyl-1H-imidazole,
[76] 1-methyl-2-{α-methyl-α-[3-(N-piperidyl)propoxy]-4-(trifluoromethyl)benzyl}-1H-imidazole,
[77] 2-{α-butyl-2-chloro-α-[3-(dimethylamino)propoxy)benzyl}-1-methyl-1H-imidazole,
[78] 2-{α-butyl-3,4-dichloro-α-[3-(dimethylamino)propoxy]benzyl}-1-methyl-1H-imidazole,
[79] 2-{3,4-dichloro-α-[3-(dimethylamino)propoxy]-α-methylbenzyl}-1-methyl-1H-imidazole,
[80] 2-{3,4-dichloro-α-[3-(dimethylamino)propoxy]benzyl}-1-methyl-1H-imidazole,
[81] 2-{α-cyclohexyl-3,4-dichloro-α-[3-(dimethylamino)propoxy]benzyl}-1-methyl-1H-imidazole,
[82] 2-{4-chloro-α-[3-(dimethylamino)propoxy)-α-methylbenzyl}-α-[2-(N-piperidyl)ethyl]-1H-imidazole,
[83] 2-{4-chloro-α-[3-(dimethylamino)propoxy]-α-methylbenzyl}-1-[2-(N-piperidyl) propyl]-1H-imidazole,
[84] 2-{4-chloro-α-[3-(dimethylamino)propoxyl-α-(N-methyl-4-piperidyl)benzyl}-1-methyl-1H-imidazole,
[85] 1-butyl-5-{α-[3-(dimethylamino)propoxylbenzyl}-1H-pyrazole,
[86] 1-butyl-5-{4-chloro-α-[3-(dimethylaminb)propoxy]-α-methylbenzyl}-1H-pyrazole,
[87] 5-{α-[3-(dimethylamino)propoxy)benzyl)-1-methyl-1H-pyrazole,
[88] 5-{α-[3-(dimethylamino)propoxy]-α-methylbenzyl}-1-methyl-1H-pyrazole,
[89] 1,3-dimethyl-5-{α-[3-(dimethylamino)propoxy)-α-methylbenzyl}-1H-pyrazole,
[90] 1,3-dimethyl-5-{α-[3-(dimethylamino)propoxy]benzyl}-1H-pyrazole,
[91] 5-{α-[3-(dimethylamino)propoxy]-2-methylbenzyl}-1-methyl-1H-pyrazole,
[92] 5-chloro-5-{4-chloro-α-[3-(dimethylamino)propoxy)benzyl}-1-methyl-1H-pyrazole,
[93] 1-methyl-5-{α-[3-(N-piperidyl)propoxy]benzyl}-1H-pyrazole,
[94] 1-methyl-5-{α-[3-(N-pyrrolidinyl)propoxy]benzyl}-1H-pyrazole,
[95] 4-{4-chloro-α-[2-(dimethylamino)ethoxy]benzyl}-1-methyl-1H-pyrazole,
[96] 4-{4-chloro-α-[2-(dimethylamino)ethoxy]-α-methylbenzyl}-1-methyl-1H-pyrazole,
[97] 4-{4-chloro-α-[2-(N-propyl-2-piperidyl)ethoxy]benzyl}-1-methyl-1H-pyrazole,
[98] 4-{4-chloro-α-[2-(N-methyl-2-piperidyl)ethoxy]benzyl}-1-methyl-1H-pyrazole,
[99] 4-{4-chloro-α-[2-(N-ethyl-2-piperidyl)ethoxy]benzyl}-1-methyl-1H-pyrazole,
[100] 4-{4-chloro-α-[2-(diisopropylamino)ethoxy]benzyl}-1-methyl-1H-pyrazole,
[101] 4-{4-chloro-(-[2-(N-methyl-2-pyrrolidinyl)ethoxy]benzyl}-1-methyl-1H-pyrazole,
[102] 4-{α-[3-(dimethylamino)propoxy]benzyl}-1-methyl-1H-pyrazole,
[103] 4-{4-chloro-α-[3-(N-morpholinyl)propoxy]benzyl}-1-methyl-1H-pyrazole,
[104] 4-{4-chloro-α-[3-(N-pyrrolidinyl)propoxy]benzyl}-1-methyl-1H-pyrazole,
[105] 2-(α-hydroxybenzyl)-1H-imidazole,
[106] 2-(4-chloro-α-hydroxybenzyl)-1H-imidazole,
[107] 2-(4-chloro-α-hydroxybenzyl)-1-methyl-1H-imidazole,
[108] 2-(3-chloro-α-hydroxybenzyl)-1-methyl-1H-imidazole,
[109] 2-(4-fluoro-α-hydroxybenzyl)-1-methyl-1H-imidazole,
[110] 2-[α-hydroxy-3-(trifluoromethyl)benzyl]-1-methyl-1H-imidazole,
[111] 2-[α-hydroxy-4-(trifluoromethyl)benzyl)-1-methyl-1H-imidazole,
[112] 2-(α-hydroxy-3,4,5-trimethoxybenzyl)-1-methyl-1H-imidazole,
[113] 2-(3,4-dichloro-α-hydroxybenzyl)-1-methyl-1H-imidazole,
[114] 1-butyl-2-[α-hydroxy-4-(trifluoromethyl)benzyl]-1H-imidazole,
[115] 1-butyl-2-(3,4-dichloro-α-hydroxybenzyl)-1H-imidazole,
[116] 1-butyl-2-(4-chloro-α-hydroxybenzyl)-1H-imidazole,
[117] 1-butyl-2-(α-hydroxy-3,4,5-trimethoxybenzyl)-1H-imidazole,
[118] 1-dodecyl-2-(α-hydroxy-3,4,5-trimethoxybenzyl)-1H-imidazole,
[119] 2-(α-butyl-3-chloro-α-hydroxybenzyl)-1-methyl-1H-imidazole,
[120] 2-(3-chloro-α-hydroxy-α-methylbenzyl)-1-methyl-1H-imidazole,
[121] 2-(4-chloro-α-hydroxy-α-methylbenzyl)-1-methyl-1H-imidazole,
[122] 2-[4-chloro-α-hydroxy-α-(N-methyl-4-piperidyl)benzyl]-1-methyl-1H-imidazole,
[123] 2-(4-chloro-α-ethyl-α-hydroxybenzyl)-1-methyl-1H-imidazole,
[124] 2-(α-butyl-4-chloro-α-hydroxybenzyl)-1-methyl-1H-imidazole,
[125] 2-(α-cyclohexyl-4-chloro-α-hydroxybenzyl)-1-methyl-1H-imidazole,
[126] 2-(2-chloro-α-hydroxy-α-methylbenzyl)-1-methyl-1H-imidazole,
[127] 2-(α-butyl-2-chloro-α-hydroxybenzyl)-1-methyl-1H-imidazole,
[128] 2-[α-hydroxy-α-methyl-3-(trifluoromethyl)benzyl]-1-methyl-1H-imidazole,
[129] 2-[α-butyl-α-hydroxy-3-(trifluoromethyl)benzyl]-1-methyl-1H-imidazole,
[130] 2-[α-cyclohexyl-α-hydroxy-3-(trifluoromethyl)benzyl)-1-methyl-1H-imidazole,
[131] 2-[α-hydroxy-α-methyl-4-(trifluoromethyl)benzyl]-1-methyl-1H-imidazole,
[132] 2-(4-fluoro-α-hydroxy-α-methylbenzyl)-1-methyl-1H-imidazole,
[133] 2-(α-hydroxy-α-methyl-4-methoxybenzyl)-1-methyl-1H-imidazole,
[134] 2-(3,4-dichloro-α-hydroxy-α-methylbenzyl)-1-methyl-1H-imidazole,
[135] 2-(α-butyl-3,4-dichloro-α-hydroxybenzyl)-1-methyl-1H-imidazole,
[136] 2-(α-cyclohexyl-3,4-dichloro-α-hydroxybenzyl)-1-methyl-1H-imidazole,
[137] 2-(α-hydroxy-α-methyl-3,4,5-trimethoxybenzyl)-1-methyl-1H-imidazole,
[138] 1-butyl-2-(4-chloro-α-hydroxy-α-methylbenzyl)-1H-imidazole,
[139] 1-butyl-2-(α-butyl-4-chloro-α-hydroxybenzyl]-1H-imidazole,
[140] 1-butyl-2-[4-chloro-α-hydroxy-α-(N-methyl-4-piperidyl)benzyl]-1H-imidazole,
[141] 1-butyl-2-(α-butyl-α-hydroxy-3,4,5-trimethoxybenzyl)-1H-imidazole,
[142] 1-butyl-2-(α-butyl-2-chloro-α-hydroxybenzyl)-1H-imidazole,
[143] 1-butyl-2-[α-ethyl-α-hydroxy-3-(trifluoromethyl)benzyl]-1H-imidazole,
[144] 1-butyl-2-(α-butyl-2,4-dichloro-α-hydroxybenzyl)-1H-imidazole,
[145] 2-(4-chloro-α-hydroxy-α-methylbenzyl)-1-[2-(N-piperidyl)ethyl]-1H-imidazole,
[146] 2-(4-chloro-α-hydroxy-α-methylbenzyl)-1-(3-dimethylaminopropyl)-1H-imidazole,
[147] 2-(α-butyl-α-hydroxy-3,4,5-trimethoxybenzyl)-1-dodecyl-1H-imidazole,
[148] 1-benzyl-2-[α-butyl-α-hydroxy-3-(trifluoromethyl)benzyl]-1H-imidazole,
[149] 1-benzyl-2-(4-chloro-α-hydroxy-α-methylbenzyl)-1H-imidazole,
[150] 1-(2-cyanoethyl)-2-(4-chloro-α-hydroxybenzyl)-1H-imidazole,
[151] 1-(3-aminopropyl)-2-(4-chloro-α-hydroxybenzyl)-1H-imidazole,
[152] 3-[2-(3-chloro-α-hydroxybenzyl)-1H-imidazole-1-yl]propanoic acid,
[153] 2-(4-chloro-α-hydroxybenzyl)-1-(3-hydroxypropyl)-1H-imidazole,
[154) 3-[2-(3-chloro-α-hydroxybenzyl)-1H-imidazole-1-yl]methyl-propanoate,
[155] 2-(α-hydroxybenzyl)-1-(3-hydroxypropyl)-1H-imidazole,
[156] 2-(α-hydroxy-4-methylbenzyl)-1-(3-hydroxypropyl)-1H-imidazole,
[157] 2-(α-hydroxy-4-methoxybenzyl)-1-(3-hydroxypropyl)-1H-imidazole,
[158] 2-(3,4-dichloro-α-hydroxybenzyl)-1-(3-hydroxypropyl)-1H-imidazole,
[159] 3-{2-(α-hydroxybenzyl)-1H-imidazole-1-yll}-methyl propanoate,
[160] 2-(4-chloro-a-hydroxybenzyl)-1-(4-hydroxybutyl)-1H-imidazole,
[161] 1-(3-cyanopropyl)-2-(4-chloro-α-hydroxybenzyl)-1H-imidazole,
[162] 4-[2-(4-chloro-α-hydroxybenzyl)-1H-imidazole-1-yl]butanoic acid,
[163] 4-[2-(4-chloro-α-hydroxybenzyl)-1H-imidazole-1-yl]-methyl butanoate,
[164] 1-butyl-5-(α-hydroxybenzyl)-1H-pyrazole,
[165] 5-(4-chloro-α-hydroxybenzyl)-1-methyl-1H-pyrazole,
[166] 5-(α-hydroxy-3,4,5-trimethoxybenzyl)-1-methyl-1H-pyrazole,
[167] 1-butyl-5-(α-hydroxy-3,4,5-trimethoxybenzyl)-1H-pyrazole,
[168] 4-bromo-5-(α-hydroxybenzyl)-1-methyl-1H-pyrazole,
[169] 5-[α-(4-chlorophenyl)-α-hydroxybenzyl]-1-methyl-1H-pyrazole,
[170] 1-butyl-5-(4-chloro-α-hydroxy-α-methylbenzyl)-1H-pyrazole,
[171] 5-(α-hydroxy-α-methylbenzyl)-1-methyl-1H-pyrazole,
[172] 5-(α-hydroxy-α-methyl-3,4,5-trimethoxybenzyl)-1-methyl-1H-pyrazole,
[173] 1,3-dimethyl-5-(α-hydroxy-α-methylbenzyl)-1H-pyrazole,
[174] 1-butyl-5-(α-hydroxy-α-vinylbenzyl)-1H-pyrazole,
[175] 1-butyl-5-(4-chloro-α-hydroxy-α-vinylbenzyl)-1H-pyrazole,
[176] 4-chloro-5-(α-hydroxybenzyl)-1-methyl-1H-pyrazole,
[177] 5-(α-hydroxy-2-methylbenzyl)-1-methyl-1H-pyrazole,
[178] 5-(3-chloro-α-hydroxybenzyl)-1-methyl-1H-pyrazole,
[179] 5-(α-hydroxy-4-methylbenzyl)-1-methyl-1H-pyrazole,
[180] 5-(2-chloro-α-hydroxybenzyl)-1-methyl-1H-pyrazole,
[181] 5-(α-hydroxy-4-methoxybenzyl)-1-methyl-1H-pyrazole,
[182] 5-{α-[2-(dimethylamino)ethoxy]-2-thienylmethyl}-1-methyl-1H-pyrazole,
[183] 5-{α-[2-(dimethylamino)ethoxy]-2-thienylmethyl)-1-methyl-1H-pyrazole citrate,
[184] 5-{(-[2-(dimethylamino)ethoxy]-3-thienylmethyl}-1-methyl-1H-pyrazole,
[185] 2-{α-[2-(dimethylamino)ethoxy]-2-thienylmethyl}-1-methyl-1H-imidazole,
[186] 5-{α-[2-(dimethylamino)ethoxy]-3-methyl-2-thienylmethyl}-1-methyl-1H-pyrazole,
[187] 5-{α-[2-(dimethylamino)ethoxyl-5-methyl-2-thienylmethyl}-1-methyl-1H-pyrazole,
[188] 5-{5-bromo-α-[2-(dimethylamino)ethoxy]-2-thienylmethyl}-1-methyl-1H-pyrazole,
[189] 5-{4-bromo-α-[2-(dimethylamino)ethoxy]-2-thienylmethyl}-1-methyl-1H-pyrazole,
[190] 5-{α-(2-(dimethylamino)ethoxy]-α-methyl-2-thienylmethyl}-1-methyl-1H-pyrazole,
[191] 5-{α-[2-(dimethylamino)ethoxy]benzyl}-1-methyl-1H-pyrazole citrate,
[192] (±)-5-{α-[2-(dimethylamino)-1-(methyl)ethoxy]benzyl}-1-methyl-1H-pyrazole,
[193] (±)-5-{α-[2-(dimethylamino)-1-(methyl)ethoxy]benzyl}-1-methyl-1H-pyrazole,
[194] (+)-5-{α-[2-(dimethylamino)ethoxy]-2-thienylmethyl}-1-methyl-1H-pyrazole,
[195] (−)-5-{α-[2-(dimethylamino)ethoxy]-2-thienylmethyl}-1-methyl-1H-pyrazole,
[196] (+)-5-{α-[2-(dimethylamino)ethoxy]-2-thienylmethyl}-1-methyl-1H-pyrazole citrate,
[197] (−)-5-{α-[2-(dimethylamino)ethoxy]-2-thienylmethyl}-1-methyl-1H-pyrazole citrate,
[198] (+)-5-{α-[2-(dimethylamino)ethoxy]-2-thienylmethyl}-1-methyl-1H-pyrazole-D-ditoluyltartrate,
[199] (−)-5-{α-[2-(dimethylamino)ethoxyl-2-thienylmethyl}-1-methyl-1H-pyrazole D-ditoluyltartrate,
[200] (+)-5-{α-[2-(dimethylamino)ethoxy]benzyl}-1-methyl-1H-pyrazole citrate,
[201] (−)-5-{α-[2-(dimethylamino)ethoxy]benzyl)-1-methyl-1H-pyrazole citrate,
[202] 5-(α-hydroxy-2-thienylmethyl)-1-methyl-1H-pyrazole,
[203] 5-(α-hydroxy-3-methyl-2-thienylmethyl)-1-methyl-1H-pyrazole,
[204] 5-(α-hydroxy-5-methyl-2-thienylmethyl)-1-methyl-1H-pyrazole,
[205] 5-(5-bromo-α-hydroxy-2-thienylmethyl)-1-methyl-1H-pyrazole,
[206] 5-(4-bromo-α-hydroxy-2-thienylmethyl)-1-methyl-1H-pyrazole and
[207] 5-(α-hydroxy-α-methyl-2-thienylmethyl)-1-methyl-1H-pyrazole
are present.

10. Active substance combination according to claim 1, characterized in that as component (b) one or more nonsteroidal anti-inflammatory drugs are present, which are selected from the group consisting of Acemetacin, Acetylsalicylic acid, Bufexamac, Diclofenac, Diflunisal, Ethenzamide, Etofenamate, Fenbufen, Fenoprofen, Feprazone, Flobufen, Flufenamic acid, Flurbiprofen, Ibuprofen, Indomethacin, Isoxicam, Kebuzone, Ketoprofen, Ketorolac, Lonazolac, Lornoxicam, Meclofenamic acid, Mefenamic acid, Metamizol, Mofebutazone, Nabumetone, Naproxen, Niflumic acid, Oxaprozin, Oxyphenbutazone, Paracetamol, Phenidine, Phenylbutazone, Piroxicam, Propacetamol, Propyphenazone, Salicylamide, Sulindac, Tenoxicam, Tiaprofenic acid, Tolmetin, Celecoxib, Etodolac, Etoricoxib, Meloxicam, Nimesulide, Parecoxib, Rofecoxib, Valdecoxib and physiologically acceptable salts thereof.

11. Active substance combination according to claim 10, characterized in that as component (b) one or more one or more nonsteroidal anti-inflammatory drugs are present, which are selected from the group consisting of Acemetacin, Acetylsalicylic acid, Bufexamac, Diclofenac, Diflunisal, Ethenzamide, Etofenamate, Fenbufen, Fenoprofen, Feprazone, Flobufen, Flufenamic acid, Flurbiprofen, Ibuprofen, Indomethacin, Isoxicam, Kebuzone, Ketoprofen, Ketorolac, Lonazolac, Lomoxicam, Meclofenamic acid, Mefenamic acid, Metamizol, Mofebutazone, Nabumetone, Naproxen, Niflumic acid, Oxaprozin, Oxyphenbutazone, Paracetamol, Phenidine, Phenylbutazone, Piroxicam, Propacetamol, Propyphenazone, Salicylamide, Sulindac, Tenoxicam, Tiaprofenic acid, Tolmetin, Etodolac, Meloxicam, Nimesulide and physiologically acceptable salts thereof.

12. Active substance combination according to claim 10, characterized in that as component (b) one or more one or more nonsteroidal anti-inflammatory drugs are present, which are selected from the group consisting of Acemetacin, Acetylsalicylic acid, Bufexamac, Diclofenac, Diflunisal, Ethenzamide, Etofenamate, Fenbufen, Fenoprofen, Feprazone, Flobufen, Flufenamic acid, Flurbiprofen, Ibuprofen, Indomethacin, Isoxicam, Kebuzone, Ketoprofen, Ketorolac, Lonazolac, Lomoxicam, Meclofenamic acid, Mefenamic acid, Metaminzol, Mofebutazone, Nabumetone, Naproxen, Niflumic acid, Oxaprozin, Oxyphenbutazone, Paracetamol, Phenidine, Phenylbutazone, Piroxicam, Propacetamol, Propyphenazone, Salicylamide, Sulindac, Tenoxicam, Tiaprofenic acid, Tolmetin and physiologically acceptable salts thereof, preferably selected from the group consisting of Acetylsalicylic acid, Diclofenac, Ibuprofen, Naproxen and physiologically acceptable salts thereof, more preferably selected from the group consisting of Diclofenac, Ibuprofen, Naproxen and physiologically acceptable salts thereof.

13. Active substance combination according to claim 1, characterized in that the molar ratio of component (A) to component (B) is in the range of 1:10 to 10:1, preferably from 1:4 to 4:1.

14. Active substance combination according to claim 1, characterized in that component (A) and component (B) are at least partially present as a salt formed from these components.

15. Active substance combination according to claim 1, characterized in that component (A) and component (B) are present in form of a 1:1 salt.

16. Active substance combination according to claim 15, characterized in that the salt is selected from the group consisting of
(a) R-(+)-5-[α- [2-(Dimethylamino)ethoxylbenzyl]-1-methyl-1H-pyrazole naproxenate (R-(+)-Cizolirtine naproxenate),
(b) S-(−)-5-[α- [2-(Dimethylamino)ethoxy]benzyl]-1-methyl-1H-pyrazole naproxenate(S-(−)-Cizolirtine naproxenate),
(c) R-(+)-5-[α-[2-(Dimethylamino)ethoxy]benzyl]-1-methyl-1H-pyrazole diclofenacate (R-(+)-Cizolirtine diclofenacate),
(d) S-(−)-5-[α-[2-(Dimethylamino)ethoxy]benzyl]-1-methyl-1H-pyrazole diclofenacate (R-(+)-Cizolirtine diclofenacate),
(e) R-(+)-5-[α-[2-(Dimethylamino)ethoxy]benzyl]-1-methyl-1H-pyrazole S-(+)-ibuprofenate (R-(+)-Cizolirtine S-(+)-ibuprofenate) and
(f) S-(−)-5-[α-[2-(Dimethylamino)ethoxy]benzyl]-1-methyl-1H-pyrazole S-(+) ibuprofenate (R-(+)-Cizolirtine S-(+)-ibuprofenate).

17. Active substance combination according to claim 1, characterized in that it further comprises as component (C) at least one agent, which is suitable to prevent the abuse of component (A) and/or component (B).

18. Active substance combination according to claim 17, characterized in that said agent(s) of component (C) is selected from the group consisting of aversive agents and/or gelling agents.

19. Medicament comprising an active substance combination according claim 1 and optionally at least one further active substance and/or optionally at least one auxiliary substance.

20. Medicament according to claim 19 for the treatment of pain, whereby said pain is preferably selected from the group consisting of neuropathic pain, acute pain, chronic pain, post-operative pain, chronic lower back pain, cluster headaches, herpes neuralgia, phantom limb pain, central pain, dental pain, resistant pain, visceral pain, surgical pain, bone injury pain, pain during labor and delivery, pain resulting from burns, pain resulting from sunburns, post partum pains, migraine, angina pain, genitourinary tract-related pain, pain from cystitis and nociceptive pain, for the prophylaxis and/or treatment of neurogenic inflammation, for the prophylaxis and/or treatment of urinary incontinence, for the prophylaxis and/or treatment of depression, for the prophylaxis and/or treatment of inflammation and/or for the prophylaxis and/or treatment of inflammation related disorders, whereby said inflammation-related disorders may preferably be selected from the group consisting of arthritis, rheumatoid arthritis, spondyloarthropathies, gouty arthritis, osteoarthritis, systemic lupus erythematosus, juvenile arthritis, rheumatic fever, symptoms associated with influenza or other viral infections, common cold, lower back pain, neck pain, dysmenorrhea, headache, toothache, sprains, strains, myositis, neuralgia, synovitis, gout, ankylosing spondylitis, bursitis, edema, inflammations following dental procedures, inflammations following dental procedures, vascular diseases, migraine headaches, periarteritis nodosa, thyroiditis, aplastic anemia, Hodkin's disease, sclerodoma, type I diabetes, myasthenia gravis, sarcoidosis, nephrotic syndrome, Behcet's syndrome, polymyositis, gingivitis, hypersensivity, conjunctivitis, swelling ocurring after injury and myocardia ischemia, for the prophylaxis and/or treatment of asthma, for the prophylaxis and/or treatment of bronchitis, for the prophylaxis and/or treatment of tendinitis, for the prophylaxis and/or treatment of bursitis, for the prophylaxis and/or treatment of skin related conditions, whereby said skin related conditions may preferably be selected from the group consisting of psoriasis, eczema, burns and dermatitis, for the prophylaxis and/or treatment of gastrointestinal disorders, whereby said gastrointestinal disorders may preferably be selected from the group consisting of inflammatory bowel disease, Crohn's disease, gastritis, irritable bowel syndrome and ulcerative colitis, or for treatment of fever, or for the prophylaxis and/or treatment of cancer or a cancer-related disorders, whereby said cancer or related disorder may preferably be selected from the group consisting of brain cancer, bone cancer, epithelial cell-derived neoplasia (epithelial carcinoma), basal cell carcinoma, adenocarcinoma, gastrointestinal cancer, lip cancer, colon cancer, liver cancer, bladder cancer, pancreas cancer, ovary cancer, cervical cancer, lung cancer, breast cancer, skin cancer, squamous cell cancer, prostate cancer, renal cell carcinoma and other known cancers that effect epithelial cells throughout the body, for the prophylaxis and/or treatment of polyps, for the prophylaxis and/or treatment of angiogenesis mediated disorders, preferably selected from the group consisting of metastasis, corneal graft rejection, ocular neovascularization, retinal neovascularisation, diabethic retinopathy, retrolental flbroplasia, neovascular glaucoma, gastric ulcer, infantile hemaginomas, angiofibroma of the nasopharynx, avascular necrosis of the bone and endometriosis.

21. Medicament according to claim 19 for the treatment of pain, whereby said pain is preferably selected from the group consisting of neuropathic pain, acute pain, chronic pain, post-operative pain, chronic lower back pain, cluster headaches, herpes neuralgia, phantom limb pain, central pain, dental pain, resistant pain, visceral pain, surgical pain, bone injury pain, pain during labor and delivery, pain resulting from burns, pain resulting from sunburns, post partum pains, migraine, angina pain, genitourinary tract-related pain, pain from cystitis and nociceptive pain.

22. Medicament according to claim 19 for the prophylaxis and/or treatment of inflammation and/or for the prophylaxis and/or treatment of inflammation related disorders, whereby said inflammation-related disorders may preferably be selected from the group consisting of arthritis, rheumatoid arthritis, spondyloarthropathies, gouty arthritis, osteoarthritis, systemic lupus erythematosus, juvenile arthritis, rheumatic fever, symptoms associated with influenza or other viral infections, common cold, lower back pain, neck pain, dysmenorrhea, headache, toothache, sprains, strains, myositis, neuralgia, synovitis, gout, ankylosing spondylitis, bursitis, edema, inflammations following dental procedures, inflammations following dental procedures, vascular diseases, migraine headaches, periarteritis nodosa, thyroiditis, aplastic anemia, Hodkin's disease, sclerodoma, type I diabetes, myasthenia gravis, sarcoidosis, nephrotic syndrome, Behcet's syndrome, polymyositis, gingivitis, hypersensivity, conjunctivitis, swelling ocurring after injury and myocardia ischemia.

23. Use of an active substance combination according to claim 1 for the manufacture of a medicament for the treatment of pain, whereby said pain is preferably selected from the group consisting of neuropathic pain, acute pain, chronic pain, post-operative pain, chronic lower back pain, cluster headaches, herpes neuralgia, phantom limb pain, central pain, dental pain, resistant pain, visceral pain, surgical pain, bone injury pain, pain during labor and delivery, pain resulting from burns, pain resulting from sunburns, post partum pains, migraine, angina pain, genitourinary tract-related pain, pain from cystitis and nociceptive pain, for the prophylaxis and/or treatment of urinary incontinence, for the prophylaxis and/or treatment of neurogenic inflammation for the prophylaxis and/or treatment of depression, for the prophylaxis and/or treatment of inflammation and/or for the prophylaxis and/or treatment of inflammation related disorders, whereby said inflammation-related disorders may preferably be selected from the group consisting of arthritis, rheumatoid arthritis, spondyloarthropathies, gouty arthritis, osteoarthritis, systemic lupus erythematosus, juvenile arthritis, rheumatic fever, symptoms associated with influenza or other viral infections, common cold, lower back pain, neck pain, dysmenorrhea, headache, toothache, sprains, strains, myositis, neuralgia, synovitis, gout, ankylosing spondylitis, bursitis, edema, inflammations following dental procedures, inflammations following dental procedures, vascular diseases, migraine headaches, periarteritis nodosa, thyroiditis, aplastic anemia, Hodkin's disease, sclerodoma, type I diabetes, myasthenia gravis, sarcoidosis, nephrotic syndrome, Behcet's syndrome, polymyositis, gingivitis, hypersensivity, conjunctivitis, swelling ocurring after injury and myocardia ischemia, for the prophylaxis and/or treatment of asthma, for the prophylaxis and/or treatment of bronchitis, for the prophylaxis and/or treatment of tendinitis, for the prophylaxis and/or treatment of bursitis, for the prophylaxis and/or treatment of skin related conditions, whereby said skin related conditions may preferably be selected from the group consisting of psoriasis, eczema, burns and dermatitis, for the prophylaxis and/or treatment of gastrointestinal disorders, whereby said gastrointestinal disorders may preferably be selected from the group consisting of inflammatory bowel disease, Crohn's disease, gastritis, irritable bowel syndrome and ulcerative colitis, or for treatment of fever, or for the prophylaxis and/or treatment of cancer or a cancer-related disorders, whereby said cancer or related disorder may preferably be selected from the group consisting of brain cancer, bone cancer, epithelial cell-derived neoplasia (epithelial carcinoma), basal cell carcinoma, adenocarcinoma, gastrointestinal cancer, lip cancer, colon cancer, liver cancer, bladder cancer, pancreas cancer, ovary cancer, cervical cancer, lung cancer, breast cancer, skin cancer, squamous cell cancer, prostate cancer, renal cell carcinoma and other known cancers that effect epithelial cells throughout the body, for the prophylaxis and/or treatment of polyps, for the prophylaxis and/or treatment of angiogenesis mediated disorders, preferably selected from the group consisting of metastasis, comeal graft rejection, ocular neovascularization, retinal neovascularisation, diabethic retinopathy, retrolental fibroplasia, neovascular glaucoma, gastric ulcer, infantile hemaginomas, angiofibroma of the nasopharynx, avascular necrosis of the bone and endometriosis.

24. Use according to claim 23 for the manufacture of a medicament for the treatment of pain, whereby said pain is preferably selected from the group consisting of neuropathic pain, acute pain, chronic pain, post-operative pain, chronic lower back pain, cluster headaches, herpes neuralgia, phantom limb pain, central pain, dental pain, resistant pain, visceral pain, surgical pain, bone injury pain, pain during labor and delivery, pain resulting from burns, pain resulting from sunburns, post partum pains, migraine, angina pain, genitourinary tract-related pain, pain from cystitis and nociceptive pain.

25. Use according to claim 23 for the manufacture of a medicament for the prophylaxis and/or treatment of inflammation and/or for the prophylaxis and/or treatment of inflammation related disorders, whereby said inflammation-related disorders may preferably be selected from the group consisting of arthritis, rheumatoid arthritis, spondyloarthropathies, gouty arthritis, osteoarthritis, systemic lupus erythematosus, juvenile arthritis, rheumatic fever, symptoms associated with influenza or other viral infections, common cold, lower back pain, neck pain, dysmenorrhea, headache, toothache, sprains, strains, myositis, neuralgia, synovitis, gout, ankylosing spondylitis, bursitis, edema, inflammations following dental procedures, inflammations following dental procedures, vascular diseases, migraine headaches, periarteritis nodosa, thyroiditis, aplastic anemia, Hodkin's disease, sclerodoma, type I diabetes, myasthenia gravis, sarcoidosis, nephrotic syndrome, Behcet's syndrome, polymyositis, gingivitis, hypersensivity, conjunctivitis, swelling ocurring after injury and myocardia ischemia.

26. Pharmaceutical formulation comprising an active substance combination according to claim 1 and optionally at least one further active substance and/or optionally at least one auxiliary.

27. Pharmaceutical formulation according to claim 26, characterized in that it is suitable for oral or parenteral administration, preferably for oral, intravenous, intraperitoneal, intramuscular, subcutaneous, intrathekal, rectal, transdermal, transmucosal or nasal administration.

28. Pharmaceutical formulation for oral administration according to claim 27, characterized in that it is in the form of a tablet, a drageé, a capsule, drops, a gel, juice, sirup, solution or suspension.

29. Pharmaceutical formulation for oral administration according to claim 27, characterized in that is in form of multiparticulates, preferably pellets or granules, optionally compressed into a tablet, filled into a capsule or suspended in a suitable liquid.

30. Pharmaceutical formulation for oral administration according to claim 27, characterized in that it comprises at least one enteric coating.

31. Pharmaceutical formulation according to claim 26 characterized in that it comprises component (A) and/or component (B) at least partially in a sustained-release form.

32. Pharmaceutical formulation according to claim 31, characterized in that the sustained release is achieved by at least one coating or matrix comprising at least one sustained-release material.

33. Pharmaceutical formulation according to claim 32, characterized in that the sustained-release material is based on an optionally modified, water-insoluble, natural, semisynthetic or synthetic polymer, or a natural, semisynthetic or synthetic wax or fat or fatty alcohol or fatty acid, or on a mixture of at least two of these afore mentioned components.

34. Pharmaceutical formulation according to claim 33, characterized in that the water-insoluble polymer is based on an acrylic resin, which is preferably selected from the group of poly(meth)acrylates, poly(C₁4dialkylamino (Ci-4alkyl (meth)acrylates and/or copolymers thereof or a mixture of at least two of the afore-mentioned polymers.

35. Pharmaceutical formulation according to claim 33, characterized in that the water-insoluble polymers are cellulose derivatives, preferably alkyl cellulose, particularly preferably ethyl cellulose, or cellulose esters.

36. Pharmaceutical formulation according to claim 33, characterized in that the wax is camauba wax, beeswax, glycerol monostearate, glycerol monobehenate, glycerol ditripalmitostearate, microcrystalline wax or a mixture of at least two of these components.

37. Pharmaceutical formulation according to claim 33, characterized in that the polymers have been used in combination with one or more plasticizers.

38. Pharmaceutical formulation according to claim 31, characterized in that it comprises component (A) and/or (B) in immediate-release form as well as in sustained release form.
